(19) United States
(12) Patent Application Publication
(10) Pub. No.: US 2026/0102416 A1
(43) Pub. Date: Apr. 16, 2026

(54) COMPOSITION OF MATTER FOR TREATING AT A MEDICAL CONDITION AND METHODS OF USE AND MANUFACTURE THEREOF

(71) Applicant: TLCO Holdings Inc., Playa Del Rey, CA (US)

(72) Inventors: Joseph Spindler, Los Angeles, CA (US); Michael William Randel, Lakebay, WA (US)

(73) Assignee: TLCO Holdings Inc., Playa Del Rey, CA (US)

(21) Appl. No.: 18/913,483

(22) Filed: Oct. 11, 2024

(51) Int. Cl. A61K 31/675; A61K 31/4709; A61K 36/07

(52) U.S. Cl. CPC A61K 31/675; A61K 31/4709; A61K 36/07; A61K 2236/37; A61K 2236/51; A61K 2236/53

(57) ABSTRACT

A composition of matter for treating a medical condition and methods of use and manufacture related thereto include providing a first extract comprising a first dosage of at least a psilocybin-based compound, providing a second extract comprising a second dosage of lion's mane, and providing a third dosage of cilostazol, combining the first extract and the second extract to form a first mixture, homogenizing the first mixture, and adding the third dosage of cilostazol to the first mixture to form a second mixture, thereby producing the composition of matter.

### FIELD OF THE INVENTION

The present invention generally relates to the field of therapeutics. In particular, the present invention is directed to composition of matter for treating a medical condition and methods of use and manufacture thereof.

### BACKGROUND

The synergistic effect between psychedelic mushrooms and other agents is presently unclear.

### SUMMARY OF THE DISCLOSURE

Psychedelic mushrooms, commonly referred to as “magic mushrooms”, are a group of fungi containing psychoactive compounds. These mushrooms have been used for centuries in various cultural and spiritual rituals due to their mind-altering effects. Recent studies suggest that psychedelic mushrooms, under proper dosages, may have potential therapeutic benefits for certain neurological conditions. However, the legality of psychedelic mushrooms varies globally, with many countries classifying them as controlled substances due to their potent effects and potential for misuse. Many aspects regarding the medicinal properties of psychedelic mushrooms and active ingredients contained therein remain to be explored.

In an aspect, a composition of matter for treating at least a medical condition pertaining to a subject is described. Composition includes a plurality of active ingredients, wherein the plurality of active ingredients includes a first dosage of at least a psilocybin-based compound, a second dosage of lion's mane, and a third dosage of cilostazol.

In another aspect, a method for producing a composition of matter for treating at least a medical condition pertaining to a subject is described. Method includes providing plurality of active ingredients, including providing a first extract comprising first dosage of at least a psilocybin-based compound, providing a second extract comprising second dosage of lion's mane, and providing a third dosage of cilostazol, combining the first extract and the second extract to form a first mixture, homogenizing the first mixture, and adding the third dosage of cilostazol to the first mixture to form a second mixture, thereby producing the composition of matter.

In another aspect, a method for treating at least a medical condition pertaining to a subject is described. Method includes administering to a subject a composition of matter including plurality of active ingredients, wherein the plurality of active ingredients includes first dosage of at least a psilocybin-based compound, second dosage of lion's mane, and third dosage of cilostazol.

In another aspect, a composition of matter for treating at least a medical condition pertaining to a subject is described. Composition is produced by providing plurality of active ingredients, including providing first extract comprising first dosage of at least a psilocybin-based compound, providing second extract comprising second dosage of lion's mane, and providing third dosage of cilostazol, combining the first extract and the second extract to form first mixture, homogenizing the first mixture, and adding the third dosage of cilostazol to the first mixture to form second mixture, thereby producing the composition of matter.

These and other aspects and features of nonlimiting embodiments of the present invention will become apparent to those skilled in the art upon review of the following description of specific nonlimiting embodiments of the invention in conjunction with the accompanying drawings.

### BRIEF DESCRIPTION OF THE DRAWINGS

For the purpose of illustrating the invention, the drawings show aspects of one or more embodiments of the invention. However, it should be understood that the present invention is not limited to the precise arrangements and instrumentalities shown in the drawings, wherein:

FIG. 1 is a schematic illustration of an exemplary embodiment of a composition of matter for treating at least a medical condition pertaining to a subject and elements related thereto;

FIG. 2 is a flow diagram of an exemplary embodiment of a method for manufacturing the composition of matter;

FIG. 3 is a flow diagram of an exemplary embodiment of a method for providing a first extract including a first dosage of at least a psilocybin-based compound;

FIG. 4 is a flow diagram of an exemplary embodiment of a method for providing a second extract including a second dosage of lion's mane; and

FIG. 5 is a flow diagram of an exemplary embodiment of a method for treating the at least a medical condition pertaining to the subject.

The drawings are not necessarily to scale and may be illustrated by phantom lines, diagrammatic representations and fragmentary views. In certain instances, details that are not necessary for an understanding of the embodiments or that render other details difficult to perceive may have been omitted.

### DETAILED DESCRIPTION

At a high level, aspects of the present disclosure are directed to a composition of matter for treating at least a medical condition pertaining to a subject and methods of its use and manufacture. Composition includes a plurality of active ingredients, wherein the plurality of active ingredients includes a first dosage of at least a psilocybin-based compound, a second dosage of lion's mane, and a third dosage of cilostazol; and a matrix. Psilocybin-based compound may include psilocybin or psilocin. In one or more embodiments, first dosage may be between 0.1 mg and 10 mg. In one or more embodiments, first dosage may be less than or equal to 0.1 mg. In one or more embodiments, second dosage may be between 5 mg and 1500 mg. In one or more embodiments, third dosage may be between 25 mg and 200 mg. In one or more embodiments, composition may include 0.5 mg psilocybin, 250 mg lion's mane, and 50 mg cilostazol. In one or more embodiments, composition may be administered in a tablet form or a capsule form or incorporated in an edible. In one or more embodiments, composition may be administered three times per week.

Aspects of the present disclosure can be used to relieve symptoms of medical conditions such as neurological conditions. Aspects of the present disclosure may allow for manufacturing of therapeutics from natural products in nature. Exemplary embodiments illustrating aspects of the present disclosure are described below in the context of several specific examples.

To facilitate the understanding of this invention, a number of terms are defined below and throughout the disclosure. Unless otherwise defined, all technical and scientific terms used herein have the same meaning as commonly understood by one of ordinary skill in the art to which this invention belongs. The terminology herein is used to describe specific embodiments of the invention, but their usage does not limit the invention, except as outlined in the claims.

It is understood that the acts described below are meant as a general overview and demonstration of an exemplary method, and that the method may include different and/or additional acts as described herein or otherwise.

While the present invention will be described as having particular configurations disclosed herein, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains.

It is to be understood that any aspect and/or element of any embodiment of the method(s) described herein or otherwise may be combined in any way to form additional embodiments of the method(s) all of which are within the scope of the method(s).

Where a process is described herein, those of ordinary skill in the art will appreciate that the process may operate without any user intervention. In another embodiment, the process includes some human intervention (e.g., a step is performed by or with the assistance of a human).

For the purposes of this disclosure, including the claims, the phrase “at least some” means “one or more” and includes the case of only one. Thus, e.g., the phrase “at least some ABCs” means “one or more ABCs” and includes the case of only one ABC.

For the purposes of this disclosure, including the claims, the term “at least one” should be understood as meaning “one or more” and therefore includes both embodiments that include one or multiple components. Furthermore, dependent claims that refer to independent claims that describe features with “at least one” have the same meaning, both when the feature is referred to as “the” and “the at least one”.

For the purposes of this disclosure, the term “portion” means some or all. Therefore, for example, “A portion of X” may include some of “X” or all of “X”. In the context of a conversation, the term “portion” means some or all of the conversation.

For the purposes of this disclosure, including the claims, the phrase “using” means “using at least” and is not exclusive. Thus, e.g., the phrase “using X” means “using at least X”. Unless specifically stated by use of the word “only”, the phrase “using X” does not mean “using only X”.

For the purposes of this disclosure, including the claims, the phrase “based on” means “based in part on” or “based, at least in part, on” and is not exclusive. Thus, e.g., the phrase “based on factor X” means “based in part on factor X” or “based, at least in part, on factor X”. Unless specifically stated by use of the word “only”, the phrase “based on X” does not mean “based only on X”.

In general, for the purposes of this disclosure, including the claims, unless the word “only” is specifically used in a phrase, it should not be read into that phrase.

For the purposes of this disclosure, including the claims, the phrase “distinct” means “at least partially distinct”. Unless specifically stated, distinct does not mean fully distinct. Thus, e.g., the phrase “X is distinct from Y” means that “X is at least partially distinct from Y” and does not mean that “X is fully distinct from Y”. Thus, for the purposes of this disclosure, including the claims, the phrase “X is distinct from Y” means that X differs from Y in at least some way.

It should be appreciated that the words “first”, “second”, and so on, in the description and claims, are used to distinguish or identify, and not to show a serial or numerical limitation.

Similarly, letter labels (e.g., “(A)”, “(B)”, “(C)”, and so on, or “(a)”, “(b)”, and so on) and/or numbers (e.g., “(i)”, “(ii)”, and so on) are used to assist in readability and to help distinguish or identify, and are not intended to be otherwise limiting or to impose or imply any serial or numerical limitations or orderings. Similarly, words such as “particular”, “specific”, “certain”, and “given”, in the description and claims, if used, are to distinguish or identify, and are not intended to be otherwise limiting.

For the purposes of this disclosure, including the claims, the terms “multiple” and “plurality” mean “two or more,” and include the case of “two”. Thus, e.g., the phrase “multiple ABCs” means “two or more ABCs” and includes “two ABCs”. Similarly, e.g., the phrase “multiple PQRs” means “two or more PQRs” and includes “two PQRs”.

The present invention also covers the exact terms, features, values, and ranges, etc., in case these terms, features, values, and ranges, etc., are used in conjunction with terms such as “about”, “around”, “generally”, “substantially”, “essentially”, “at least”, etc. Thus, e.g., “about 3” or “approximately 3” shall also cover exactly 3, and “substantially constant” shall also cover exactly constant.

For the purposes of this disclosure, unless stated otherwise, the terms “about” or “approximately” refer to a value that is within 10% above or below the value being described.

For the purposes of this disclosure, including the claims, singular forms of terms are to be construed as also including the plural form and vice versa, unless the context indicates otherwise. Thus, it should be noted that for the purposes of this disclosure, the singular forms “a”, “an”, and “the” include plural references unless the context clearly dictates otherwise. In other words, terms such as “a”, “an”, and “the” are not intended to refer to only a singular entity but include the general class of which a specific example may be used for illustration.

Throughout the description and claims, the terms “comprise”, “including”, “having”, “contain”, and their variations should be understood as meaning “including but not limited to” and are not intended to exclude other components unless specifically so stated.

For the purposes of this disclosure, the terms “administration” or “administering” refer to a method of giving a dosage of a compound or pharmaceutical composition to a subject.

For the purposes of this disclosure, the terms “treat”, “treating”, or “treatment” refer to administration of a compound or pharmaceutical composition for a therapeutic purpose. To “treat a disorder” or use for “therapeutic treatment” refers to administering treatment to a patient already suffering from a disease to ameliorate the disease or one or more symptoms thereof to improve the patient's condition (e.g., by reducing one or more symptoms of a neurological disorder). The term “therapeutic” includes the effect of mitigating deleterious clinical effects of certain processes (i.e., consequences of the process, rather than the symptoms of processes). In some embodiments, a compound or pharmaceutical composition may be administered as a prophylactic and/or in order to prevent a subject from having a medical condition. A process, composition, or the like described herein as being used for treatment of a condition may also be used for prevention of the condition. As used herein, “prevention” is administration of a compound or pharmaceutical composition to a subject in order to stop the subject from having a medical condition, in order to slow the onset of a medical condition, in order to reduce the likelihood that a subject gains a medical condition, or a combination thereof.

It will be appreciated that variations to the embodiments of the invention can be made while still falling within the scope of the invention. Alternative features serving the same, equivalent, or similar purpose can replace features disclosed in the specification, unless stated otherwise. Thus, unless stated otherwise, each feature disclosed represents one example of a generic series of equivalent or similar features.

Use of exemplary language, such as “for instance”, “such as”, “for example” (“e.g.,”), and the like, is merely intended to better illustrate the invention and does not indicate a limitation on the scope of the invention unless specifically so claimed.

While the invention has been described in connection with what is presently considered to be the most practical and embodiments and is further described in the examples below, it is to be understood that the invention is not to be limited to the disclosed embodiment, but on the contrary, is intended to cover various modifications and equivalent arrangements included within the spirit and scope of the appended claims.

The following description sets forth various examples along with specific details to provide a thorough understanding of claimed subject matter. It will be understood by those skilled in the art, however, that claimed subject matter may be practiced without one or more of the specific details disclosed herein. Further, in some circumstances, well-known methods, procedures, systems, and/or components have not been described in detail in order to avoid unnecessarily obscuring claimed subject matter. The illustrative embodiments described in the detailed description and claims are not meant to be limiting. Other embodiments may be utilized, and other changes may be made, without departing from the spirit or scope of the subject matter presented here. It will be readily understood that the aspects of the present disclosure, as generally described herein, can be arranged, substituted, combined, and designed in a wide variety of different configurations, all of which are explicitly contemplated and make part of this disclosure.

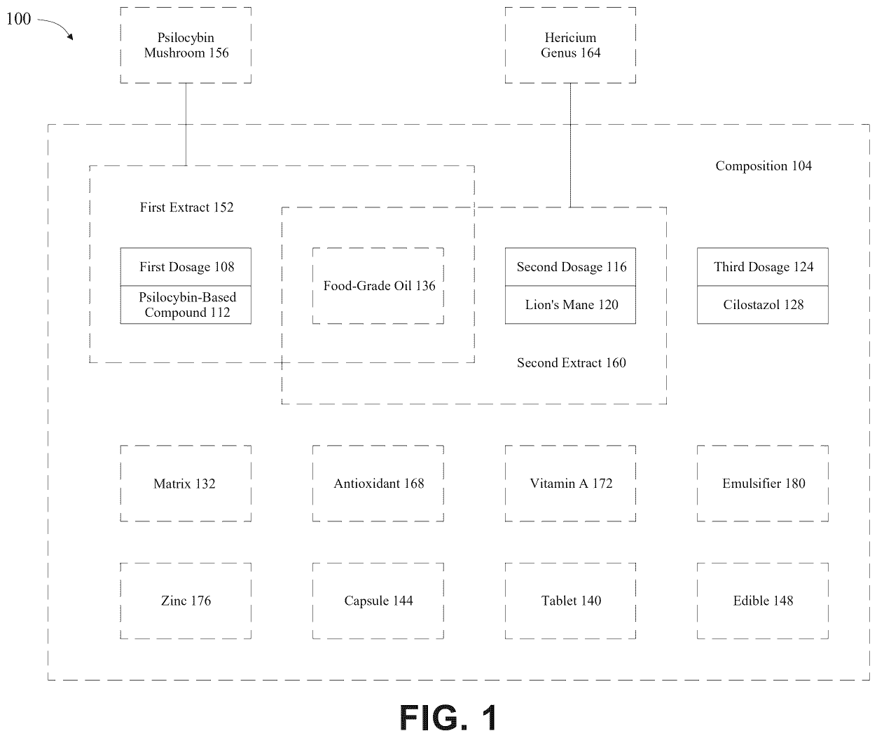

Referring now to FIG. 1, an exemplary embodiment 100 of a composition 104 for treating at least a medical condition pertaining to a subject and elements related thereto is illustrated. For the purposes of this disclosure, a “medical condition” is a disease, disorder, ailment, injury, or abnormal physical, or mental state affecting a living organism, which requires medical attention, intervention, or treatment. Medical condition may be physiological or psychological and may differentiate a subject from a healthy individual. Medical condition may include a neurological condition. For the purposes of this disclosure, a “neurological condition” is a disorder or disease that affects the nervous system, which includes the brain, spinal cord, peripheral nerves, and neuromuscular junctions. Neurological condition may encompass a wide range of medical issues characterized by a disruption of normal neural function, leading to various physiological, cognitive, and behavioral impairments. As nonlimiting examples, neurological condition may result from genetic anomalies, neurodegenerative processes, traumatic injuries, infections, autoimmune responses, vascular disorders, or metabolic imbalances.

With continued reference to FIG. 1, medical condition may include a case of pain such as pain from neuroischemic disorder. For the purposes of this disclosure, “neuroischemic disorder” is a medical condition characterized by a combination of nerve damage (i.e., neuropathy) and inadequate blood supply (i.e., ischemia) to a particular region of the body, often leading to complex clinical presentations and requires multidisciplinary management. Medical condition may include a case of motor neuron disease. For the purposes of this disclosure, “motor neuron disease” is a neurodegenerative disorder characterized by a progressive degeneration and loss of motor neurons, which are specialized nerve cells responsible for transmitting neural impulses from the central nervous system to the muscles and facilitating voluntary muscle control. The disorder encompasses a spectrum of clinical manifestations, the most prevalent being Amyotrophic Lateral Sclerosis (ALS), also known as Lou Gehrig's disease. Medical condition may include a case of spinal muscular atrophy. For the purposes of this disclosure, “spinal muscular atrophy” is a genetic disorder characterized by a progressive degeneration of alpha motor neurons in the anterior horn of the spinal cord, resulting in muscular atrophy and weakness. Medical condition may include a case of peripheral neuropathy. For the purposes of this disclosure, “peripheral neuropathy” is a pathological condition characterized by a damage or dysfunction of the peripheral nerves, which are the nerves outside the central nervous system (CNS) including the brain and spinal cord. This condition impairs the transmission of sensory, motor, and autonomic signals between the CNS and the rest of the body, resulting in a range of symptoms depending on the type and extent of nerve damage. Medical condition may include a case of fibromyalgia. For the purposes of this disclosure, “fibromyalgia” is a chronic disorder characterized by widespread musculoskeletal pain, fatigue, and tenderness in localized areas referred to as “tender points”. The condition involves a complex pathophysiology that is not fully understood but is believed to involve abnormal processing of pain signals in the CNS. Medical condition may include a case of hypoxia-ischemia injury. For the purposes of this disclosure, “hypoxia-ischemia injury” is a pathological condition that results from a combination of inadequate oxygen supply (i.e., hypoxia) and reduced blood flow (i.e., ischemia) to tissues or organs. This dual impairment leads to cellular dysfunction and death, primarily affecting highly metabolic and oxygen-dependent tissues such as the brain and heart. Medical condition may include a case of diabetic complication including diabetic polyneuropathy. For the purposes of this disclosure, “diabetic polyneuropathy” is a chronic and progressive disorder characterized by damage to multiple peripheral nerves, resulting from long-term hyperglycemia associated with diabetes mellitus. This condition manifests through a range of sensory, motor, and autonomic nerve dysfunctions, significantly impacting the patient's quality of life. Medical condition may include a case of multiple sclerosis. For the purposes of this disclosure, “multiple sclerosis” is a chronic, inflammatory, demyelinating, and neurodegenerative disease of the CNS characterized by an immune-mediated attack on myelin, the protective sheath surrounding nerve fibers. This leads to a disrupted communication between the brain and other parts of the body, resulting in a wide array of neurological symptoms and potential physical/cognitive disabilities.

With continued reference to FIG. 1, medical condition may include a case of macular degeneration. For the purposes of this disclosure, “macular degeneration” or “age-related macular degeneration (AMD)” is a medical condition that primarily affects the central part of the retina called the macula. Macula is responsible for sharp, central vision and necessary for tasks such as reading, driving, and recognizing faces. As the macula deteriorates, individuals experience a loss of central vision, although peripheral vision typically remains unaffected. Macular degeneration may include dry macular degeneration (non-exudative), which is a more common type that accounts for approximately 85-90% of macular degeneration cases. It is characterized by a thinning of the macula and the accumulation of tiny yellow deposits known as drusen under the retina. Over time, the presence of drusen can lead to atrophy of the retinal cells, resulting in a gradual loss of central vision. Macular degeneration may include wet macular degeneration (exudative), which is a less common yet more severe type that accounts for approximately 10-15% of macular degeneration cases. It occurs when abnormal blood vessels grow under the retina and macula, leading to blood and fluid leakage. This leakage causes rapid damage to the retinal cells, leading to sudden and significant vision loss if not treated promptly. Composition 104 may increase blood flow to the eye and allow medications such as vitamin A and zinc, among other AREDS 2-compliant medications, to be delivered and absorbed by the retina. Similarly, composition 104 may facilitate a passage of medications and/or nutrients across the blood brain barrier, thereby alleviating symptoms of a neurological condition, as described above.

With continued reference to FIG. 1, additionally and/or alternatively, medical condition may include retinal conditions, anxiety, depression, concussion, post-traumatic stress disorder (PTSD), cluster headaches, addiction, smoking cessation, obsessive compulsive disorder (OCD), attention deficit disorder (ADD), small blood vessel disease, Alzheimer's disease, dementia, Parkinson's disease, Huntington's disease, stroke, spine injury, brain injury, migraine, menopause, weight loss, cancer, psychosis, nausea, inflammatory disease, rheumatoid arthritis, infection, inflammatory bowel disease, cardiovascular condition, and the like.

With continued reference to FIG. 1, composition 104 includes a plurality of active ingredients. For the purposes of this disclosure, an “active ingredient” is a chemical/pharmaceutical or mixture of chemicals/pharmaceuticals capable of creating one or more medicinal effects and/or treating one or more symptoms pertaining to a medical condition. In contrast, for the purposes of this disclosure, an “inactive ingredient” is a chemical or mixture of chemicals that is not medically effective. Inactive ingredients, such as matrices, binders, disintegrants, fillers, lubricants, and coatings, among others, as described below, may be used to stabilize one or more active ingredients, prevent their degradation over time, and preserve their functions as part of a formulation.

With continued reference to FIG. 1, plurality of active ingredients may include a first dosage 108 of at least a psilocybin-based compound 112. For the purposes of this disclosure, a “dosage” is a specific amount of a medication or drug that is prescribed for administration at one time or over a set period. Dosage is a crucial aspect of treatment plans to ensure effectiveness and minimize the risk of adverse effects. Dosage determines how much and how often a drug is taken and is tailored to individual patient needs based on factors such as age, weight, the condition being treated, and the patient's response to the medication. For the purpose of this disclosure, a “psilocybin-based compound” is a compound that bears structural similarity to psilocybin. As nonlimiting examples, psilocybin-based compound 112 may include psilocybin, psilocin, and/or the like. For the purposes of this disclosure, “psilocybin”, or 4-phosphoryloxy-N,N-dimethyltryptamine, is a naturally occurring psychedelic compound with a chemical formula of C₁₂H₁₇N₂O₄P. Psilocybin is produced by over 200 species of fungi, notably within the Psilocybe genus. Psilocybin is a prodrug to psilocin and may be metabolized vis dephosphorylation in the human body to form psilocin, its pharmacologically active form, which impacts the CNS by agonistically binding to serotonin receptors, particularly the 5-HT2A receptor. The engagement of psilocybin with neural pathways alters perception, emotion, and cognition, presenting notable implications in therapeutic contexts. Scientific studies underscore psilocybin's potential efficacy in treating various psychiatric disorders, including depression, anxiety, PTSD, and conditions of existential distress associated with terminal illness, consistent with details described above. The profound effects observed from single-dose administrations have propelled psilocybin to the forefront of psychiatric research, leading to its designation as a breakthrough therapy in mental health treatment, reflecting its significant therapeutic promise. Psilocybin mushrooms, their enzymes, and/or their indole alkaloids may counteract and reduce the toxicity of methyl phosphonates—the core structure of sarin, a potent neurotoxin used in nerve gases. Moreover, the use of psilocybin-based compound 112 including psilocin and/or psilocybin in the compositions described herein may help block the neurotoxicity effects of chemical nerve toxins and lead to protecting and healing military and civilians exposed to sarin and other neurotoxic agents. The use of psilocybin-based compound 112 may also potentially enable neurogenesis by removing agents harmful to neurological health, including but not limited to parasites such as nematodes, protozoa, pathogenic bacteria including Borrelia species and other spirochetes, and/or other infectious organisms or viruses. Additional benefits may be conferred by psilocybin-based compound 112 not only to humans challenged with diseases or neurotoxins, but also in healthy humans, including but not limited to an increased cognitive function in students, scientists, computer coders, hackers, big data specialists, mathematicians, astronomers, strategic planners (i.e., such as in the U.S. Defense Department), gamers, linguists, writers, futurists, Mensa members, athletes, soldiers, religious leaders, politicians, business leaders or anyone benefitting from increased memory, intelligence, imagination, cognition, clairvoyance, motor skills, spatial navigation, athleticism, ability to more quickly respond to and process stimuli, balance, neuroplasticity, state of mind, longevity and mental health. Various benefits of psilocybin-based compound 112 may also be expected in vertebrate animals including mammals, carnivores, omnivores, herbivores, pets including cats and dogs and companion animals, farm and produce animals, laboratory animals, zoo animals, reptiles, fish, and birds, among others. Psilocybin-based compound 112 may also be useful for bees, including those suffering from pesticides, viruses or at risk from Colony Collapse Disorder, and/or various other invertebrates.

With continued reference to FIG. 1, in one or more embodiments, first dosage 108 of at least a psilocybin-based compound 112 may be between 0.01 mg and 10 mg, e.g., 0.01 mg to 0.05 mg, 0.05 mg to 0.1 mg, 0.1 mg to 0.2 mg, 0.2 mg to 0.5 mg, 0.5 mg to 1 mg, 1 mg to 1.5 mg, 1.5 mg to 2 mg, 2 mg to 3 mg, 3 mg to 4 mg, 4 mg to 5 mg, 5 mg to 6 mg, 6 mg to 7 mg, 7 mg to 8 mg, 8 mg to 9 mg, and 9 mg to 10 mg. In some cases, first dosage 108 of at least a psilocybin-based compound 112 may be above 0.1 mg, e.g., above 0.1 mg, above 0.5 mg, above 1 mg, above 1.5 mg, above 2 mg, above 2.5 mg, above 3 mg, above 3.5 mg, above 4 mg, above 4.5 mg, above 5 mg, above 5.5 mg, above 6 mg, above 6.5 mg, above 7 mg, above 7.5 mg, above 8 mg, above 8.5 mg, above 9 mg, or above 9.5 mg.

With continued reference to FIG. 1, in some cases, first dosage 108 of at least a psilocybin-based compound 112 may be approximately 0.01 mg, approximately 0.05 mg, approximately 0.1 mg, approximately 0.25 mg, approximately 0.5 mg, approximately 0.75 mg, approximately 1 mg, approximately 1.25 mg, approximately 1.5 mg, approximately 2 mg, approximately 2.5 mg, approximately 3 mg, approximately 4 mg, approximately 5 mg, approximately 7.5 mg, or approximately 10 mg.

With continued reference to FIG. 1, in one or more embodiments, composition 104 may include between 0.1% and 99.7% by weight of first dosage of at least a psilocybin-based compound 112. As nonlimiting examples, composition 104 may include 0.1% to 0.5%, 0.5% to 1%, 1% to 1.5%, 1.5% to 2%, 2% to 2.5%, 2.5% to 3%, 3% to 3.5%, 3.5% to 4%, 4% to 4.5%, and 4.5% to 5%, 5% to 6%, 6% to 7%, 7% to 8%, 8% to 9%, 9% to 10%, 10% to 12%, 12% to 14%, 14% to 16%, 16% to 18%, 18% to 20%, 20% to 25%, 25% to 30%, 30% to 35%, 35% to 40%, 40% to 45%, and 45% to 50%, 50% to 60%, 60% to 70%, 70% to 80%, 80% to 90%, 90% to 95%, 95% to 99%, 99% to 99.5%, or 99.5% to 99.7% by weight of first dosage 108 of at least a psilocybin-based compound 112.

With continued reference to FIG. 1, in one or more embodiments, composition 104 may include greater than 0.1%, greater than 0.5%, greater than 1%, greater than 1.5%, greater than 2%, greater than 2.5%, greater than 3%, greater than 3.5%, greater than 4%, greater than 4.5%, and greater than 5% greater than 10%, greater than 15%, greater than 20%, greater than 25%, greater than 30%, greater than 35%, greater than 40%, greater than 45%, greater than 50%, greater than 60%, greater than 70%, greater than 80%, greater than 90%, greater than 95%, greater than 99%, or greater than 99.5% by weight of first dosage 108 of at least a psilocybin-based compound 112.

With continued reference to FIG. 1, composition 104 includes a second dosage 116 of lion's mane 120. For the purposes of this disclosure, “lion's mane” is a medicinally active chemical or a plurality of medicinally active chemicals extracted from a culinary and medicinal mushroom of the same name distinguished by its long, white spines resembling a mane. Lion's mane 120 may include a variety of bioactive compounds such as hericenones and erinacines, which stimulate nerve growth factor synthesis and exhibit neuroprotective properties, polysaccharides including beta-glucans that enhance immune function, as well as fatty acids and lipids that offer anti-inflammatory benefits. Lion's mane 120 has been shown to improve cognitive function, potentially aiding in the treatment of neurodegenerative diseases like Alzheimer's and Parkinson's, enhance mood, and alleviate symptoms of anxiety and depression. Its antioxidant properties further contribute to health maintenance and disease prevention. Given its wide range of health benefits, Lion's mane 120 may be potentially useful in pharmaceutical compositions to support neurological health, immune response, and mental health. Lion's mane 120 may be available in forms such as capsules, extracts, powders, teas, or the like.

With continued reference to FIG. 1, in one or more embodiments, second dosage 116 of lion's mane 120 may be between 5 mg and 1500 mg, e.g., 5 mg to 10 mg, 10 mg to 15 mg, 15 mg to 20 mg, 20 mg to 25 mg, 25 mg to 30 mg, 30 mg to 35 mg, 35 mg to 40 mg, 40 mg to 45 mg, and 45 mg to 50 mg, 50 mg to 100 mg, 100 mg to 150 mg, 150 mg to 200 mg, 200 mg to 250 mg, 250 mg to 300 mg, 300 mg to 350 mg, 350 mg to 400 mg, 400 mg to 450 mg, 450 mg to 500 mg, 500 mg to 600 mg, 600 mg to 700 mg, 700 mg to 800 mg, 800 mg to 900 mg, 900 mg to 1000 mg, 1000 mg to 1100 mg, 1100 mg to 1200 mg, 1200 mg to 1300 mg, 1300 mg to 1400 mg, or 1400 mg to 1500 mg.

With continued reference to FIG. 1, in some cases, second dosage 116 of lion's mane 120 may be approximately 5 mg, approximately 10 mg, approximately 15 mg, approximately 20 mg, approximately 25 mg, approximately 30 mg, approximately 35 mg, approximately 40 mg, approximately 45 mg, approximately 50 mg, approximately 100 mg, approximately 150 mg, approximately 200 mg, approximately 250 mg, approximately 300 mg, approximately 350 mg, approximately 400 mg, approximately 450 mg, approximately 500 mg, approximately 600 mg, approximately 700 mg, approximately 800 mg, approximately 900 mg, approximately 1000 mg, approximately 1100 mg, approximately 1200 mg, approximately 1300 mg, approximately 1400 mg, or approximately 1500 mg.

With continued reference to FIG. 1, in some cases, second dosage 116 of lion's mane 120 may be above 5 mg, above 10 mg, above 50 mg, above 100 mg, above 150 mg, above 200 mg, above 250 mg, above 300 mg, above 350 mg, above 400 mg, above 450 mg, above 500 mg, above 550 mg, above 600 mg, above 650 mg, above 700 mg, above 750 mg, above 800 mg, above 850 mg, above 900 mg, above 950 mg, above 1000 mg, above 1050 mg, above 1100 mg, above 1150 mg, above 1200 mg, above 1250 mg, above 1300 mg, above 1350 mg, above 1400 mg, or above 1450 mg.

With continued reference to FIG. 1, in one or more embodiments, composition 104 may include between 0.1% and 99.7% by weight of second dosage 116 of lion's mane 120. As nonlimiting examples, composition 104 may include 0.1% to 0.5%, 0.5% to 1%, 1% to 1.5%, 1.5% to 2%, 2% to 2.5%, 2.5% to 3%, 3% to 3.5%, 3.5% to 4%, 4% to 4.5%, and 4.5% to 5%, 5% to 6%, 6% to 7%, 7% to 8%, 8% to 9%, 9% to 10%, 10% to 12%, 12% to 14%, 14% to 16%, 16% to 18%, 18% to 20%, 20% to 25%, 25% to 30%, 30% to 35%, 35% to 40%, 40% to 45%, and 45% to 50%, 50% to 60%, 60% to 70%, 70% to 80%, 80% to 90%, 90% to 95%, 95% to 99%, 99% to 99.5%, or 99.5% to 99.7% by weight of second dosage of lion's mane.

With continued reference to FIG. 1, in one or more embodiments, composition 104 may include greater than 0.1%, greater than 0.5%, greater than 1%, greater than 1.5%, greater than 2%, greater than 2.5%, greater than 3%, greater than 3.5%, greater than 4%, greater than 4.5%, and greater than 5% greater than 10%, greater than 15%, greater than 20%, greater than 25%, greater than 30%, greater than 35%, greater than 40%, greater than 45%, greater than 50%, greater than 60%, greater than 70%, greater than 80%, greater than 90%, greater than 95%, greater than 99%, or greater than 99.5% by weight of second dosage 116 of lion's mane 120.

With continued reference to FIG. 1, in one or more embodiments, composition 104 may include a plant extract, such as without limitation a plant extract with neuro-regenerative properties, in addition to psilocybin-based compound 112 and lion's mane 120. Exemplary plant extracts with neuro-regenerative properties may include without limitation Bacopa species (e.g., Bacopa monnieri), Gotu kola (Centella asiatica), and Gingko (Gingko biloba). Additionally, plant extract may include an extract of one or more plants with anti-inflammatory properties. Such plants may include without limitation ginger (Zingiber officinale), Holy Basil (Ocimum sanctum), Hu Zhang (Polygonum cuspidatum), oregano (Origanum vulgare, Origanum onites, etc.), rosemary (Rosmarinus officinalis, Rosmarinus eriocalyx, and/or other species in the genus Rosmarinus), turmeric (Curcuma longa), green tea (Camellia sinensis), lavender (Lavandula spica and/or related species in the genus Lavandula), skullcap (Scutellaria lateriflora), and/or oat straw (Avena sativa, Avena byzantine, etc.). Moreover, plant extract may include without limitation Salvia divinorum, also known as Diviner's Sage. Additionally, and/or alternatively, plant extract may include ayahuasca, a concoction made from Banisteriopsis caapi and Psychotria species. Similarly, plant extract may be extracted from plants containing ibogaine (Tabernanthe iboga, Voacanga africana, and/or Tabernaemontana undulate, etc.), peyote (Lophophora williamsii), the seeds of morning glory (Ipomoea tricolor and/or related species) and Hawaiian baby wood rose (Argyreia nervosa), and cannabis (Cannabis sativa, Cannabis indica, and/or Cannabis ruderalis, etc.). Additionally, and/or alternatively, plant extract may include one or more nervine agents from natural products including without limitation cordycepin (or Cordyceps extracts containing such) and/or amyloban (which may be found in Lion's mane).

With continued reference to FIG. 1, composition 104 includes a third dosage 124 of cilostazol 128. For the purposes of this disclosure, “cilostazol”, or 6-[4-(1-cyclohexyl-1H-tetrazol-5-yl) butoxy]-3,4-dihydro-2 (1H)-quinolinone, is a pharmaceutical compound identified with a chemical formula of C₂₀H₂₇N₅O₂. Cilostazol 128 may function as a therapeutic agent with applications in improving vascular health and function, particularly beneficial in conditions associated with impaired blood flow and platelet aggregation. Its mechanism of action involves selective inhibition of phosphodiesterase III, leading to varied therapeutic outcomes primarily in the cardiovascular system, such as an increase in intracellular cAMP in platelets and blood vessels, resulting in an inhibition of platelet aggregation and vasodilation. Cilostazol 128 is utilized predominantly for the clinical management of intermittent claudication in individuals suffering from peripheral artery disease (PAD). Cilostazol 128 has also been explored for its potential to inhibit the progression of symptoms in secondary stroke prevention. Studies have suggested that its antithrombotic and vasodilatory properties may reduce the risk of recurrent stroke. Cilostazol 128 may also have applications in improving outcomes in patients with coronary artery disease, due to its effects on blood circulation.

With continued reference to FIG. 1, in one or more embodiments, third dosage 124 of cilostazol 128 may be between 25 mg and 200 mg, e.g., 25 mg to 30 mg, 30 mg to 35 mg, 35 mg to 40 mg, 40 mg to 45 mg, 45 mg to 50 mg, 50 mg to 55 mg, 55 mg to 60 mg, 60 mg to 65 mg, 65 mg to 70 mg, 70 mg to 75 mg, 75 mg to 80 mg, 80 mg to 85 mg, 85 mg to 90 mg, 90 mg to 95 mg, 95 mg to 100 mg, 100 mg to 105 mg, 105 mg to 110 mg, 110 mg to 115 mg, 115 mg to 120 mg, 120 mg to 125 mg, 125 mg to 130 mg, 130 mg to 135 mg, 140 mg to 145 mg, 145 mg to 150 mg, 150 mg to 155 mg, 155 mg to 160 mg, 160 mg to 165 mg, 165 mg to 170 mg, 170 mg to 175 mg, 175 mg to 180 mg, 180 mg to 185 mg, 185 mg to 190 mg, 190 mg to 195 mg, or 195 mg to 200 mg.

With continued reference to FIG. 1, in some cases, third dosage 124 of cilostazol 128 may be approximately 25 mg, approximately 30 mg, approximately 35 mg, approximately 40 mg, approximately 45 mg, approximately 50 mg, approximately 55 mg, approximately 60 mg, approximately 65 mg, approximately 70 mg, approximately 75 mg, approximately 80 mg, approximately 85 mg, approximately 90 mg, approximately 95 mg, approximately 100 mg, approximately 105 mg, approximately 110 mg, approximately 115 mg, approximately 120 mg, approximately 125 mg, approximately 130 mg, approximately 135 mg, approximately 140 mg, approximately 145 mg, approximately 150 mg, approximately 155 mg, approximately 160 mg, approximately 165 mg, approximately 170 mg, approximately 175 mg, approximately 180 mg, approximately 185 mg, approximately 190 mg, approximately 195 mg, and approximately 200 mg.

With continued reference to FIG. 1, in some cases, third dosage 124 of cilostazol 128 may be above 25 mg, above 30 mg, above 35 mg, above 40 mg, above 45 mg, above 50 mg, above 55 mg, above 60 mg, above 65 mg, above 70 mg, above 75 mg, above 80 mg, above 85 mg, above 90 mg, above 95 mg,

With continued reference to FIG. 1, in one or more embodiments, composition 104 may include between 0.1% and 99.7% by weight of third dosage 124 of cilostazol 128. As nonlimiting examples, composition 104 may include 0.1% to 0.5%, 0.5% to 1%, 1% to 1.5%, 1.5% to 2%, 2% to 2.5%, 2.5% to 3%, 3% to 3.5%, 3.5% to 4%, 4% to 4.5%, and 4.5% to 5%, 5% to 6%, 6% to 7%, 7% to 8%, 8% to 9%, 9% to 10%, 10% to 12%, 12% to 14%, 14% to 16%, 16% to 18%, 18% to 20%, 20% to 25%, 25% to 30%, 30% to 35%, 35% to 40%, 40% to 45%, and 45% to 50%, 50% to 60%, 60% to 70%, 70% to 80%, 80% to 90%, 90% to 95%, 95% to 99%, 99% to 99.5%, or 99.5% to 99.7% by weight of third dosage 124 of cilostazol 128.

With continued reference to FIG. 1, in one or more embodiments, composition 104 may include greater than 0.1%, greater than 0.5%, greater than 1%, greater than 1.5%, greater than 2%, greater than 2.5%, greater than 3%, greater than 3.5%, greater than 4%, greater than 4.5%, and greater than 5% greater than 10%, greater than 15%, greater than 20%, greater than 25%, greater than 30%, greater than 35%, greater than 40%, greater than 45%, greater than 50%, greater than 60%, greater than 70%, greater than 80%, greater than 90%, greater than 95%, greater than 99%, or greater than 99.5% by weight of third dosage 124 of cilostazol 128.

With continued reference to FIG. 1, in some embodiments, a subject may receive between 200 mg and 3000 mg of Cilostazol per day. For example, a subject may receive more than one dose of Cilostazol and/or a composition containing Cilostazol such that a total daily dose is between 200 mg and 3000 mg of Cilostazol. In some embodiments, a subject may receive between 500 mg and 3000 mg of Cilostazol per day. In some embodiments, a subject may receive between 100 mg and 200 mg of Cilostazol per day.

With continued reference to FIG. 1, in one or more embodiments, composition 104 may include one or more phosphodiesterase (PDE) inhibitors. For the purposes of this disclosure, a “phosphodiesterase inhibitor” or “PDE inhibitor” is an agent acting on specific phosphodiesterase enzymes in target cells. Phosphodiesterase inhibitors may include FDA-approved phosphodiesterase inhibitors that may be used for the management of chronic obstructive pulmonary disease, erectile dysfunction, pulmonary arterial hypertension, psoriasis, psoriatic arthritis, and/or atopic dermatitis (AD). In some embodiments, a phosphodiesterase inhibitor is a phosphodiesterase III inhibitor. One role of phosphodiesterase is to separate the phosphate group from a target cell and decrease the level of cyclic adenosine monophosphate (cAMP) or cyclic guanosine monophosphate (cGMP). Inhibition of phosphodiesterase may prevent the degradation of cAMP or cGMP, which may result in smooth muscle relaxation, vasodilatory, and/or bronchodilatory effects. Among common phosphodiesterase inhibitors, sildenafil is a common agent targeting PDE-5 and is FDA-approved in 1998 for erectile dysfunction (ED). The use of sildenafil may not be limited to the treatment of ED and may extend to pulmonary arterial hypertension (PAH) in adults (which obtained FDA approval in 2005). The use of sildenafil in pediatric populations for PAH has not received approval and is considered off-label use. Vardenafil and avanafil are also PDE-5 inhibitors approved for treating ED. Tadalafil is a PDE-5 inhibitor approved to treat benign prostatic hyperplasia (BPH) and ED. As both conditions may coincide in the later durations of life, tadalafil may be used as monotherapy for its therapeutic use to treat both conditions. Cilostazol, as described above, is a PDE-3 inhibitor that may reversibly inhibit platelets from aggregation and may be used to treat intermittent claudication from peripheral arterial disease (PAD). Dipyridamole is also a PDE-3 inhibitor and may be used for postoperative prophylaxis for thrombosis, but its action on the PDE-3 enzyme may be relatively weak. Dipyridamole may also be used as a diagnostic tool as a pharmacologic nuclear stress test. Dypriadamole may increase perfusion to the myocardium, revealing reversible and irreversible ischemic areas in the myocardial muscle. Milrinone and amrinone may also inhibit PDE-3 enzymes, but their actions target heart cells, increasing cAMP in the myocardium and producing a positive inotropic effect. Both agents may be used short-term for decompensated heart failure, as a therapy for greater durations may precipitate ventricular arrhythmias. Roflumilast is a PDE-4 inhibitor that may act on target cells of lung tissue and may be used for treatment of chronic obstructive pulmonary disease (COPD). Other PDE-4 inhibitors include apremilast and crisaborole. Their uses may not be pulmonary and may include without limitation treating psoriasis, psoriatic arthritis (PA), and/or AD due to their action of decreasing pro-inflammatory chemokines. PDE-4 inhibitors may decrease pro-inflammatory mediators and cause bronchodilation by inhibiting the degradation of cAMP. Theophylline is a non-specific PDE inhibitor with weak action on phosphodiesterase enzymes and has been approved for symptomatic relief of COPD. Pentoxifylline is a xanthine analog and is a non-selective phosphodiesterase inhibitor. It obtained FDA approval in 1984 for treating muscle pain caused by intermittent claudication in chronic peripheral arterial disease. Anagrelide is a PDE-3 inhibitor approved for the treatment of thrombocythemia caused by myeloproliferative disorders. Ibudilast is a non-selective PDE inhibitor that has not secured approval by the FDA for its use. Ibudilast may causes dilation of bronchioles and has been used to treat asthma in Japan since 1989. Ibudilast may be able to cross the blood-brain barrier, exert anti-inflammatory actions in the CNS, and may be a promising agent for treating inflammatory neurological disorders. Caffeine, one of the most popularly utilized psychoactive compounds used globally, may also inhibit phosphodiesterase as one of its three mechanisms of action. It is labeled and generally recognized as safe (GRAS) by the FDA since 1958. Caffeine is a CNS stimulant that may be used for treating fatigue and/or drowsiness. Caffeine may also be used for enhancing memory, cognition, and/or performance. Caffeine citrate has been used to treat apnea of prematurity in neonates gestation age from twenty-two weeks to less than thirty-three weeks and received approval in 1999 for its use. PDE-3 inhibitors may include cilostazol, dipyridamole, milrinone, and amrinone, among others. PDE-4 inhibitors may include roflumilast, apremilast, and crisaborole, among others. PDE-5 inhibitors may include sildenafil, tadalafil, vardenafil, and avanafil, among others. Nonspecific PDE inhibitors may include theophylline, ibudilast, and/or the like. FDA-approved uses include treatment of ED using sildenafil, tadalafil, vardenafil, and/or avanafil; treatment of benign BPH using tadalafil; treatment of PAH using sildenafil and/or tadalafil; treatment of PA using apremilast; treatment of psoriasis: apremilast; treatment of COPD using roflumilast and/or theophylline; treatment of PAD using cilostazol and/or pentoxifylline; treatment of postoperative thromboembolic prophylaxis using dipyridamole; treatment of decompensated cardiac failure using milrinone and/or amrinone; treatment of AD using crisaborole; treatment of thrombocythemia using anagrelide; and treatment of neonatal apnea using caffeine citrate.

With continued reference to FIG. 1, in one or more embodiments, composition 104 may include a matrix 132. For the purposes of this disclosure, a “matrix” is a host including one or more inactive ingredients, as described above, in which one or more active ingredients are incorporated, integrated, encapsulated, protected, and/or stabilized. In some cases, composition 104 may include between 0.1% and 99.7% by weight of matrix 132. As nonlimiting examples, composition 104 may include 0.1% to 0.5%, 0.5% to 1%, 1% to 1.5%, 1.5% to 2%, 2% to 2.5%, 2.5% to 3%, 3% to 3.5%, 3.5% to 4%, 4% to 4.5%, and 4.5% to 5%, 5% to 6%, 6% to 7%, 7% to 8%, 8% to 9%, 9% to 10%, 10% to 12%, 12% to 14%, 14% to 16%, 16% to 18%, 18% to 20%, 20% to 25%, 25% to 30%, 30% to 35%, 35% to 40%, 40% to 45%, and 45% to 50%, 50% to 60%, 60% to 70%, 70% to 80%, 80% to 90%, 90% to 95%, 95% to 99%, 99% to 99.5%, or 99.5% to 99.7% by weight of matrix 132.

With continued reference to FIG. 1, matrix may include any matrix or filler deemed suitable by a person of ordinary skill in the art, upon reviewing the entirety of this disclosure, such as without limitation food-grade oil 136, lactose, microcrystalline cellulose, starch, calcium phosphate, calcium carbonate, sucrose, maltodextrin, mannitol, sorbitol, and sodium chloride, among others. For the purposes of this disclosure, a “food-grade oil” is an oil or mixture of oils that is safe and suitable for use in food preparation and consumption. Food-grade oil 136 must meet specific purity standards that ensure they do not contain harmful levels of any substances that could be toxic when ingested. Food-grade oils 136 are processed, packaged, and stored in ways that comply with food safety regulations to prevent contamination. Food-grade oils 136 may be derived from various sources, including plants, seeds, nuts, and even some fish, and may be used in cooking, baking, and as dressings or condiments. Each type of food-grade oil 136 may have different characteristics in terms of flavor, smoking point, and nutritional content, making some food-grade oils 136 more suitable for certain types of cooking and food preparation than others. Nonlimiting examples of food-grade oils 136 may include olive oil (Olea europaea), canola oil (Brassica napus), sunflower oil (Helianthus annuus), soybean oil (Glycine max), peanut oil (Arachis hypogaea), sesame oil (Sesamum indicum), corn oil (Zea mays), coconut oil (Cocos nucifera), palm oil (Elaeis guineensis), palm kernel oil (Elaeis guineensis), avocado oil (Persea americana), grapeseed oil (Vitis vinifera), walnut oil (Juglans regia), hazelnut oil (Corylus avellana), almond oil (Prunus dulcis), macadamia nut oil (Macadamia integrifolia), pistachio oil (Pistacia vera), flaxseed oil (Linum usitatissimum), hemp seed oil (Cannabis sativa), pumpkin seed oil (Cucurbita pepo), rice bran oil (Oryza sativa), safflower oil (Carthamus tinctorius), mustard oil (Brassica juncea), camelina oil (Camelina sativa), cottonseed oil (Gossypium spp.), chia seed oil (Salvia hispanica), perilla oil (Perilla frutescens), moringa oil (Moringa oleifera), argan oil (Argania spinosa), pecan oil (Carya illinoinensis), medium chain triglycerides oil, vegetable oil, fish oil, butter, shortening, margarine, lard, and the like.

With continued reference to FIG. 1, in one or more embodiments, composition 104 may be in a form of tablet 140. For the purposes of this disclosure, a “tablet” is a solid dosage form designed for oral administration, created by compressing active ingredients with inactive ingredients like binders, disintegrants, fillers, lubricants, and coatings, among others. Tablets come in various shapes and sizes, and may include coatings to mask taste, protect the stomach, or control release. Types of tablets 140 may include without limitation immediate-release, extended-release, chewable, effervescent, sublingual, and buccal, among others. Tablets 140 may offer convenience, stability, precise dosages, and improved patient compliance, making them a widely used and effective method for delivering medications. In one or more embodiments, composition 104 may be in a form of capsule 144. For the purposes of this disclosure, a “capsule” is a pharmaceutical dosage form including a gelatin or polymer shell designed to encapsulate active ingredients and inactive ingredients. Capsules 144 may contain powders, granules, liquids, or semi-solids and may be engineered for immediate, delayed, or sustained release. Capsules 144 may offer precise dosing, protect contents from environmental factors, and enhance patient compliance by masking taste and odor. Capsules 144 may be available in hard and soft forms, providing versatility in drug formulation and manufacturing, making them a widely used and effective method for delivering various therapeutic agents. In one or more embodiments, composition 104 may be in a form of an edible 148. For the purposes of this disclosure, an “edible” is a substance or product that is safe and suitable for human consumption. Edible 148 may provide essential nutrients or deliver therapeutic agents through oral ingestion. Edibles 148 must meet safety standards. Edibles 148 often offer nutritional value or therapeutic benefits and are usually palatable to ensure compliance. Common examples of edibles 148 may include without limitation fruits, vegetables, nuts, seeds, meat, dairy products, processed foods, vitamin gummies, medicinal lozenges, cannabis-infused gummies, chocolates, and liquid supplements. Edibles 148 may be designed to support overall health, provide necessary nutrients, or treat medical conditions effectively. Additional details will be described below in this disclosure.

With continued reference to FIG. 1, in one or more embodiments, composition 104 may include at least a food-grade oil 136, as described above. In some cases, first dosage 108 of at least a psilocybin-based compound 112 may be dissolved in at least a food-grade oil 136, as a first extract. In some cases, second dosage 116 of lion's mane 120 may be dissolved in at least a food-grade oil 136, as a second extract. In some cases, at least a psilocybin-based compound 112 and/or lion's mane 120 may be extracted from one or more raw materials (including dehydrated or non-dehydrated mushroom material). In some cases, third dosage 124 of cilostazol 128 may be dissolved in at least a food-grade oil 136. In some cases, third dosage 124 of cilostazol may be in a powder form. For the purposes of this disclosure, an “extract” is a concentrated substance derived from a raw material using solvents or mechanical processes, often containing potent active ingredients. In the food industry, extracts like vanilla, almond, and herbal extracts may enhance flavor and nutritional value in cooking and food processing. In the pharmaceutical industry, extracts like ginseng, echinacea, and green tea may be used in medications and supplements for their therapeutic benefits. Extracts may be characterized by their concentration, purity, standardization, and versatility, ensuring consistent potency and quality for various applications. For the purposes of this disclosure, a “raw material” is a material that contains one or more useful ingredients but is unprocessed or not fully processed. Raw materials may include without limitation plants, animal tissues, fungi, among others.

With continued reference to FIG. 1, at least a food-grade oil 136 may include or be included in a first extract 152 of at least a psilocybin mushroom 156. For the purposes of this disclosure, “psilocybin mushrooms”, commonly known as magic mushrooms, are a group of fungi that contain psychoactive compounds including psilocybin and psilocin. These substances are known for their ability to induce psychedelic experiences characterized by visual and mental hallucinations, changes in perception, and altered states of consciousness, as described above. Psilocybin mushrooms 156 are typically ingested orally, either dried or fresh, brewed into a tea, or incorporated into foods. The use of psilocybin mushrooms 156 spans centuries, with evidence of their ceremonial and medicinal use in various cultures, particularly among indigenous peoples of the Americas. Modern interest often focuses on their potential therapeutic benefits, with research studies exploring their use in treating depression, anxiety, PTSD, and addiction. Legally, psilocybin mushrooms 156 are classified as a Schedule I drug in the United States and are similarly controlled under international law, meaning they are currently regarded as having a high potential for abuse and no accepted medical use in treatment in some jurisdictions. However, recent shifts in research and drug policy are leading to a reevaluation of these mushrooms' therapeutic potential, and some regions have begun to decriminalize or legalize their use for medical purposes. Psilocybin mushroom 156 may belong to genera such as Psilocybe, Gymnopilus, Panaeolus, Copelandia, Pluteus, Inocybe, Pholiotina, and Galerina, among others. In addition to psilocybin and psilocin, compounds naturally produced by the mycelium of psilocybin mushrooms 156 and/or their mycelium may include bacocystin, norbacocystin, N,N-dimethyltryptamine, 5-hydroxytryptamine (serotonin), and/or 5-hydroxytryptophan.

With continued reference to FIG. 1, at least a food-grade oil 136 may include a second extract 160 of a lion's mane mushroom, i.e., a mushroom of Hericium genus 164. Hericium genus 164 may include one or more species such as Hericium erinaceus, Hericium americanum, Hericium coralloides, Hericium abietis, Hericium alpestre, Hericium cirrhatum, Hericium rajendrae, and Hericium flagellum, among others. Hericium erinaceus, Hericium americanum, Hericium coralloides, Hericium abietis, and Hericium alpestre may be found in America. Hericium erinaceus, Hericium coralloides, Hericium abietis, Hericium cirrhatum, and Hericium rajendrae may be found in Asia. Hericium erinaceus, Hericium coralloides, Hericium alpestre, Hericium cirrhatum, and Hericium flagellum may be found in Europe. These species are recognized for their unique appearance and potential medicinal benefits, with Hericium erinaceus being the most commonly utilized in traditional and modern health practices.

With continued reference to FIG. 1, in one or more embodiments, composition 104 may further include elements such as an antioxidant 168, vitamin A 172, or zinc 176, among others. For the purposes of this disclosure, an “antioxidant” is a chemical that is capable of reacting with and neutralizing oxidizing species, which include free radicals with unpaired valence electrons, that cause cellular damage in the body. Antioxidants 168 play a critical role in protecting cells from oxidative stress, which is linked to various chronic diseases such as heart disease, diabetes, and cancer. Antioxidants 168 may also support immune defense, reduce chronic inflammation, and promote healthy aging by maintaining cellular health and integrity. Nonlimiting examples of common antioxidants include vitamins C and E, beta-carotene, selenium, and phytochemicals like flavonoids and polyphenols found in a variety of fruits, vegetables, nuts, and grains, among others. While antioxidants 168 may be crucial for reducing oxidative stress, the effectiveness of antioxidant supplements in disease prevention is still subject to ongoing research and may vary based on individual health conditions and diet. As a nonlimiting example, antioxidant 168 such as Vitamin C may improve the bioavailability of psilocybin by increasing the activity of the liver enzyme CYP450, which is involved in the metabolism of psilocybin. This may increase the level of psilocybin that reaches the brain, which may lead to longer lasting and stronger experience for a user. For the purposes of this disclosure, “vitamin A” is a fat/oil-soluble vitamin essential for numerous bodily functions, including maintaining healthy vision, supporting the immune system, promoting cell growth, and ensuring proper functioning of the heart, lungs, and kidneys. Vitamin A 172 may exist in two primary forms: preformed vitamin A (retinol and retinyl esters) found in animal products, and provitamin A carotenoids (such as beta-carotene) found in plant-based foods. Vitamin A 172 plays a critical role in maintaining the health of the retina, particularly in low-light conditions, by forming the pigment rhodopsin. Vitamin A 172 also aids in the maintenance of skin and mucous membrane integrity, which serves as a barrier to infections. Additionally, vitamin A 172 is crucial for embryonic development and cellular communication, making it vital for overall growth and development. For the purposes of this disclosure, “zinc” is an essential trace element that plays a critical role in numerous biological functions in the human body. Zinc 176 is a key component of over 300 enzymes, facilitating various biochemical reactions, including DNA synthesis, protein synthesis, cell division, and repair. Zinc 176 is vital for maintaining a healthy immune system, as it aids in the development and function of immune cells, such as T lymphocytes. Zinc 176 also supports wound healing by promoting cell growth and regeneration. Furthermore, zinc 176 is crucial for proper growth and development during pregnancy, childhood, and adolescence. Zinc 176 contributes to the sense of taste and smell, and it is necessary for proper insulin function and glucose metabolism. Zinc 176 also has antioxidant properties, helping to protect cells from damage caused by free radicals, as described above. Overall, zinc 176 is indispensable for maintaining overall health, supporting a wide array of physiological processes, and ensuring the proper functioning of numerous bodily systems.

With continued reference to FIG. 1, composition 104 may further include at least an emulsifier 180. For the purposes of this disclosure, an “emulsifier” is a chemical capable of stabilizing emulsions by reducing the surface tension between immiscible liquids including oil and water. Emulsifiers 180 are essential in various industries for creating homogeneous mixtures. In food production, emulsifiers 180 such as lecithin and mono- and diglycerides enhance texture and extend product shelf life. In cosmetics, emulsifiers 180 such as polysorbates facilitate an even distribution of ingredients in products such as lotions. Pharmaceuticals employ emulsifiers 180 such as Polysorbate 80 to improve the bioavailability of drugs, particularly those that are hydrophobic and unlikely to dissolve in water. Additionally, industrial applications leverage emulsifiers 180 in products such as lubricants and paints to ensure stability and performance. Emulsifiers 180 are pivotal in enhancing product stability, usability, and efficacy across multiple sectors, underscoring their critical role in modern manufacturing and formulation processes. Emulsifier 180 may stabilize various components within composition 104 as a homogeneous mixture and prevent them from segregating into layers.

With continued reference to FIG. 1, composition 104 may include one or more excipients. For the purposes of this disclosure, an “excipient” an inactive substance used as a carrier or medium for one or more active ingredients in a formulation. Excipients themselves may not have any therapeutic effect but serve various functional roles that are essential for the stability, efficacy, and/or manufacturability of a drug product. Excipients are often used to aid in a manufacturing process, enhance a product's stability, improve the taste or appearance of a product, facilitate drug absorption, and/or control the release of an active ingredient. Exemplary excipients may include without limitation binders, fillers/diluents, disintegrants, lubricants, coatings, preservatives, stabilizers, and/or the like. Exemplary excipients may include without limitation acidifying agents (acetic acid, glacial acetic acid, citric acid, fumaric acid, hydrochloric acid, diluted hydrochloric acid, malic acid, nitric acid, phosphoric acid, diluted phosphoric acid, sulfuric acid, and tartaric acid, etc.); alkalizing agents (ammonia solution, ammonium carbonate, diethanolamine, diisopropanolamine, potassium hydroxide, sodium bicarbonate, sodium borate, sodium carbonate, sodium hydroxide, and trolamine, etc.); antifoaming agents (dimethicone, simethicone, and/or the like); antimicrobial preservatives (benzalkonium chloride, benzalkonium chloride solution, benzethonium chloride, benzoic acid, benzyl alcohol, butylparaben, cetylpyridinium chloride, chlorobutanol, chlorocresol, cresol, dehydroacetic acid, ethylparaben, methylparaben, methylparaben sodium, phenol, phenylethyl alcohol, phenylmercuric acetate, phenylmercuric nitrate, potassium benzoate, potassium sorbate, propylparaben, propylparaben sodium, sodium benzoate, sodium dehydroacetate, sodium propionate, ascorbic acid, thimerosal, thymol, and/or the like); antioxidants (ascorbic acid, ascorbyl palmitate, butylated hydroxyanisole, butylated hydroxytoluene, hypophosphorous acid, monothioglycerol, propyl gallate, sodium formaldehyde sulfoxylate, sodium metabisulfite, sodium thiosulfate, sulfur dioxide, tocopherol, tocopherols excipient, and/or the like); buffering agents (acetic acid, ammonium carbonate, ammonium phosphate, boric acid, citric acid, lactic acid, phosphoric acid, potassium citrate, potassium metaphosphate, potassium phosphate monobasic, sodium acetate, sodium citrate, sodium lactate solution, dibasic sodium phosphate, monobasic sodium phosphate, and/or the like); chelating agents (edetate disodium, ethylenediaminetetraacetic acid and salts, edetic acid, and/or the like); coating agents (sodium carboxymethylcellulose, cellulose acetate, cellulose acetate phthalate, ethylcellulose, gelatin, pharmaceutical glaze, hydroxypropyl cellulose, hydroxypropyl methylcellulose, hydroxypropyl methylcellulose phthalate, methacrylic acid copolymer, methylcellulose, polyvinyl acetate phthalate, shellac, sucrose, titanium dioxide, carnauba wax, microcrystalline wax, zein, and/or the like); colorants (caramel, red, yellow, black or blends, ferric oxide, and/or the like); complexing agents (ethylenediaminetetraacetic acid (EDTA) and salts thereof, edetic acid, gentisic acid ethanolamide, oxyquinoline sulfate, and/or the like); desiccants (calcium chloride, calcium sulfate, silicon dioxide, and/or the like); emulsifying and/or solubilizing agents (acacia, cholesterol, diethanolamine (adjunct), glyceryl monostearate, lanolin alcohols, mono- and di-glycerides, monoethanolamine (adjunct), lecithin, oleic acid (adjunct), oleyl alcohol (stabilizer), poloxamer, polyoxyethylene 50 stearate, polyoxyl 35 castor oil, polyoxyl 40 hydrogenated castor oil, polyoxyl 10 oleyl ether, polyoxyl 20 cetostearyl ether, polyoxyl 40 stearate, polysorbate 20, polysorbate 40, polysorbate 60, polysorbate 80, diacetate, monostearate, sodium lauryl sulfate, sodium stearate, sorbitan monolaurate, sorbitan monooleate, sorbitan monopalmitate, sorbitan monostearate, stearic acid, trolamine, emulsifying wax, and/or the like); filtering aids (powdered cellulose, purified siliceous earth, and/or the like); flavors and perfumes (anethole, benzaldehyde, ethyl vanillin, menthol, methyl salicylate, monosodium glutamate, orange flower oil, peppermint, peppermint oil, peppermint spirit, rose oil, stronger rose water, thymol, tolu balsam tincture, vanilla, vanilla tincture, vanillin, and/or the like); humectants (glycerol, hexylene glycol, sorbitol, and/or the like); plasticizers (castor oil, diacetylated monoglycerides, diethyl phthalate, glycerol, mono- and di-acetylated monoglycerides, propylene glycol, triacetin, triethyl citrate, and/or the like); polymers (cellulose acetate, alkyl celluloses, hydroxyalkyl, acrylic polymers and copolymers, and/or the like); solvents (acetone, alcohol, diluted alcohol, amylene hydrate, benzyl benzoate, butyl alcohol, carbon tetrachloride, chloroform, corn oil, cottonseed oil, ethyl acetate, glycerol, hexylene glycol, isopropyl alcohol, methyl alcohol, methylene chloride, methyl isobutyl ketone, mineral oil, peanut oil, propylene carbonate, sesame oil, water for injection, sterile water for injection, sterile water for irrigation, purified water, and/or the like); sorbents (powdered cellulose, charcoal, purified siliceous earth, and/or the like); carbon dioxide sorbents (barium hydroxide lime, soda lime, and/or the like); stiffening agents (hydrogenated castor oil, cetostearyl alcohol, cetyl alcohol, cetyl esters wax, hard fat, paraffin, polyethylene excipient, stearyl alcohol, emulsifying wax, white wax, yellow wax, and/or the like); suspending and/or viscosity-increasing agents (acacia, agar, alginic acid, aluminum monostearate, bentonite, purified bentonite, magma bentonite, carbomer, carboxymethylcellulose calcium, carboxymethylcellulose sodium, carboxymethylcellulose sodium carrageenan, microcrystalline and carboxymethylcellulose sodium cellulose, dextrin, gelatin, guar gum, hydroxyethyl cellulose, hydroxypropyl cellulose, hydroxypropyl methylcellulose, magnesium aluminum silicate, methylcellulose, pectin, polyethylene oxide, polyvinyl alcohol, povidone, alginate, silicon dioxide, colloidal silicon dioxide, sodium alginate, tragacanth, xanthan gum, and/or the like); sweetening agents (aspartame, dextrates, dextrose, excipient dextrose, fructose, mannitol, saccharin, calcium saccharin, sodium saccharin, sorbitol, solution sorbitol, sucrose, compressible sugar, confectioner's sugar, syrup, and/or the like); surfactants (simethicone, etc.); tablet binders (acacia, alginic acid, sodium carboxymethylcellulose, microcrystalline cellulose, dextrin, ethylcellulose, gelatin, liquid glucose, guar gum, hydroxypropyl methylcellulose, methylcellulose, polyethylene oxide, povidone, pregelatinized starch, syrup, and/or the like); tablet and/or capsule diluents (calcium carbonate, dibasic calcium phosphate, tribasic calcium phosphate, calcium sulfate, microcrystalline cellulose, powdered cellulose, dextrates, dextrin, dextrose excipient, fructose, kaolin, lactose, mannitol, sorbitol, starch, pregelatinized starch, sucrose, compressible sugar, confectioner's sugar, and/or the like); tablet disintegrants (alginic acid, microcrystalline cellulose, croscarmellose sodium, crospovidone, polacrilin potassium, sodium starch glycolate, starch, pregelatinized starch, and/or the like); tablet and/or capsule lubricants (calcium stearate, glyceryl behenate, magnesium stearate, light mineral oil, sodium stearyl fumarate, stearic acid, purified stearic acid, talc, hydrogenated vegetable oil, zinc stearate, and/or the like); thickening agents (gelatin having a bloom strength of 50-100, and/or the like); tonicity agent (dextrose, glycerol, mannitol, potassium chloride, sodium chloride, and/or the like); flavored and/or sweetened vehicle (aromatic elixir, compound benzaldehyde elixir, iso-alcoholic elixir, peppermint water, sorbitol solution, syrup, tolu balsam syrup, and/or the like); oleaginous vehicle (almond oil, corn oil, cottonseed oil, ethyl oleate, isopropyl myristate, isopropyl palmitate, mineral oil, light mineral oil, myristyl alcohol, octyl dodecanol, olive oil, peanut oil, persic oil, sesame oil, soybean oil, squalane, and/or the like); vehicle: solid carrier (sugar spheres, etc.); sterile vehicle (bacteriostatic water for injection, bacteriostatic sodium chloride injection, and/or the like); viscosity-increasing agents (see suspending agent above); water-repelling agents (cyclomethicone, dimethicone, simethiconevehicle, and/or the like); and/or solubilizing agent (benzalkonium chloride, benzethonium chloride, cetylpyridinium chloride, docusate sodium, nonoxynol 9, nonoxynol 10, octoxynol 9, poloxamer, polyoxyl 35 castor oil, polyoxyl 40, hydrogenated castor oil, polyoxyl 50 stearate, polyoxyl 10 oleyl ether, polyoxyl 20, cetostearyl ether, polyoxyl 40 stearate, polysorbate 20, polysorbate 40, polysorbate 60, polysorbate 80, sodium lauryl sulfate, sorbitan monolaurate, sorbitan monooleate, sorbitan monopalmitate, sorbitan monostearate, tyloxapol, and/or the like). It is worth noting that this list is not meant to be exhaustive, but instead merely representative of the types of excipients that may be used for the invention described herein.

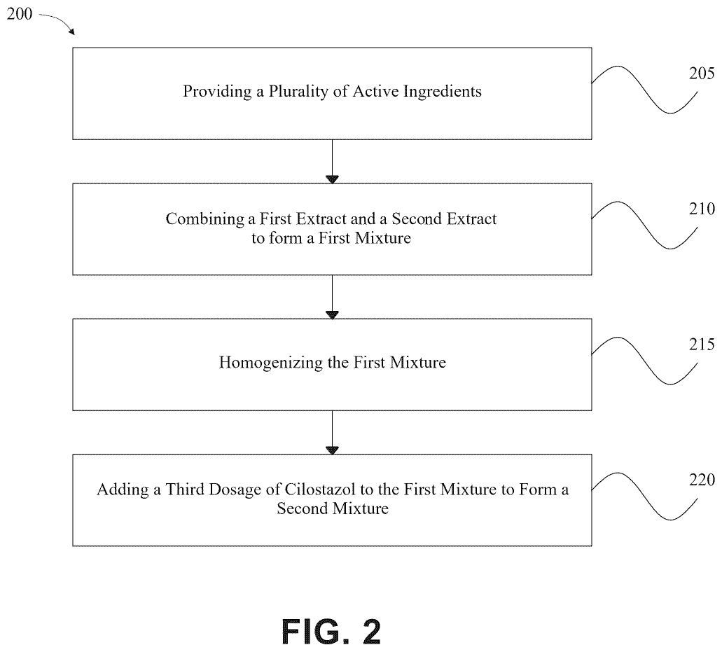

Referring now to FIG. 2, an exemplary embodiment of a method 200 of producing composition 104 for treating at least a medical condition pertaining to a subject is described. Composition 104 may include any embodiment of composition 104 described above without limitation. At step 205, method 200 includes providing a plurality of active ingredients. Specifically, step 205 includes providing first extract 152 including first dosage 108 of at least a psilocybin-based compound 112, providing second extract 160 including second dosage 116 of lion's mane 120, and providing third dosage 124 of cilostazol 128. Additional details will be provided below in this disclosure.

With continued reference to FIG. 2, at step 210, method 200 includes combining first extract 152 and second extract 160 to form a first mixture. This step may be implemented with reference to details described in this disclosure and without limitation.

With continued reference to FIG. 2, at step 215, method 200 includes homogenizing first mixture. This step may be implemented with reference to details described in this disclosure and without limitation.

With continued reference to FIG. 2, at step 220, method 200 includes adding third dosage 124 of cilostazol 128 to first mixture to form a second mixture. This step may be implemented with reference to details described in this disclosure and without limitation. In one or more embodiments, third dosage 124 of cilostazol 128 may be added in a powdered form.

With continued reference to FIG. 2, in one or more embodiments, method 200 may further include combining plurality of active ingredients with matrix 132. Matrix 132 may include any matrix 132 of any quantity described in this disclosure without limitation.

With continued reference to FIG. 2, in one or more embodiments, method 200 may further include forming composition 104 into a tablet form. In one or more embodiments, method 200 may further include forming composition 104 into a or a capsule form. In one or more embodiments, method 200 may further include infusing an edible using second mixture, details of which may be consistent with any detail described above and/or disclosed in U.S. patent application Ser. No. 18/425,334 (attorney docket number 1567-007USU1), filed on Jan. 29, 2024, entitled “CONSUMABLES INFUSED WITH COMPOUNDS FROM CANNABIS, NATURALLY OCCURRING SUBSTANCES, AND MUSHROOMS”, the entirety of which is incorporated herein by reference.

With continued reference to FIG. 2, in one or more embodiments, method 200 may further include combining plurality of active ingredients with antioxidant 168, consistent with details described above. In one or more embodiments, method 200 may further include combining plurality of active ingredients with vitamin A 172, consistent with details described above. In one or more embodiments, method 200 may further include combining plurality of active ingredients with zinc 176, consistent with details described above. In one or more embodiments, method 200 may further include combining plurality of active ingredients with at least an emulsifier 180, consistent with details described above.

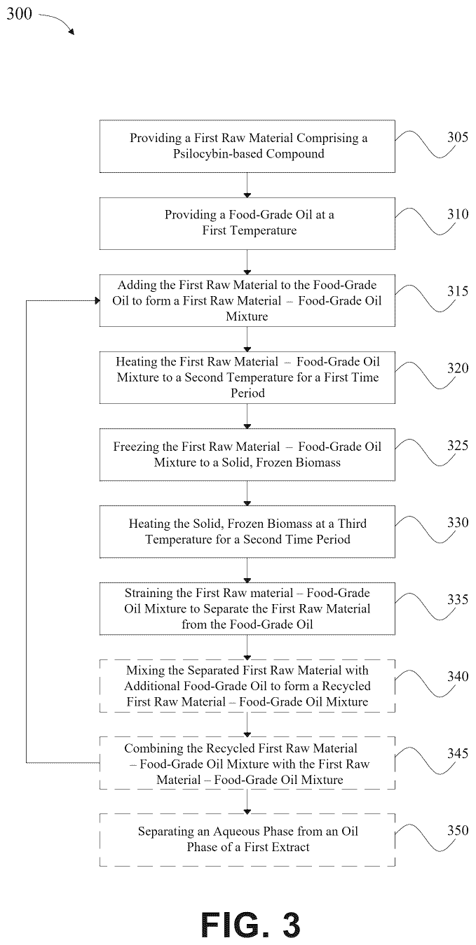

Referring now to FIG. 3, an exemplary embodiment of a method 300 for producing first extract 152 including first dosage 108 of at least a psilocybin-based compound 112 is illustrated. Implementation of method 300 may be consistent with any details disclosed in U.S. patent application Ser. No. 17/949,126 (attorney docket number 1567-004USU1), filed on Sep. 20, 2022, entitled “MUSHROOM COMPOUNDS INFUSED CONSUMABLES AND NON-CONSUMABLES”, the entirety of which is incorporated herein by reference.

With continued reference to FIG. 3, at step 305, method 300 includes providing a first raw material including at least a psilocybin-based compound 112. In one or more embodiments, first raw material may include at least a psilocybin mushroom 156, consistent with details described above in this disclosure. In some cases, at least a psilocybin mushroom may belong to a genus such as Psilocybe, Gymnopilus, Panaeolus, Copelandia, Pluteus, Inocybe, Pholiotina, and Galerina, among others. In one or more embodiments, first raw material may include a portion of a psilocybin mushroom 156, such as a cap of psilocybin mushroom 156.

With continued reference to FIG. 3, at step 310, method 300 includes providing food-grade oil 136 at a first temperature. This step may be implemented with reference to details described above in this disclosure and without limitation. In one or more embodiments, first temperature may be between 200° F.-240° F., such as 200° F.-210° F., 210° F.-220° F., 220° F.-230° F., 230° F.-240° F., 200° F.-205° F., 205° F.-210° F., 210° F.-215° F., 215° F.-220° F., 220° F.-225° F., 225° F.-230° F., 230° F.-235° F., or 235° F.-240° F. In some cases, first temperature may be approximately 200° F., approximately 205° F., approximately 210° F., approximately 215° F., approximately 220° F., approximately 225° F., approximately 230° F., approximately 235° F., or approximately 240° F.

With continued reference to FIG. 3, at step 315, method 300 includes adding at least a portion of first raw material to food-grade oil 136 to form a first raw material-food-grade oil mixture. This step may be implemented with reference to details described above in this disclosure and without limitation.

With continued reference to FIG. 3, at step 320, method 300 includes heating first raw material-food-grade oil mixture to a second temperature for a first time period. This step may be implemented with reference to details described above in this disclosure and without limitation. In one or more embodiments, second temperature may be between 200° F.-240° F., such as 200° F.-210° F., 210° F.-220° F., 220° F.-230° F., 230° F.-240° F., 200° F.-205° F., 205° F.-210° F., 210° F.-215° F., 215° F.-220° F., 220° F.-225° F., 225° F.-230° F., 230° F.-235° F., or 235° F.-240° F. In some cases, second temperature may be approximately 200° F., approximately 205° F., approximately 210° F., approximately 215° F., approximately 220° F., approximately 225° F., approximately 230° F., approximately 235° F., or approximately 240° F. In one or more embodiments, first time period may be between 4 hours and 9 hours, such as 4 hours-4.5 hours, 4.5 hours-5 hours, 5 hours-5.5 hours, 5.5 hours-6 hours, 6 hours-6.5 hours, 6.5 hours-7 hours, 7 hours-7.5 hours, 7.5 hours-8 hours, 8 hours-8.5 hours, or 8.5 hours-9 hours. In some cases, first time period may be approximately 4 hours, approximately 4.5 hours, approximately 5 hours, approximately 5.5 hours, approximately 6 hours, approximately 6.5 hours, approximately 7 hours, approximately 7.5 hours, approximately 8 hours, approximately 8.5 hours, or approximately 9 hours.

With continued reference to FIG. 3, at step 325, method 300 includes freezing first raw material-food-grade oil mixture into a solid, frozen biomass. This step may be implemented with reference to details described above in this disclosure and without limitation.

With continued reference to FIG. 3, at step 330, method 300 includes heating solid, frozen biomass at a third temperature for a second time period, thereby releasing at least a psilocybin-based compound 112 from first raw material into food-grade oil 136. This step may be implemented with reference to details described above in this disclosure and without limitation. In one or more embodiments, third temperature may be between 150° F.-200° F., such as 150° F.-160° F., 160° F.-170° F., 170° F.-180° F., 180° F.-190° F., 190° F.-200° F., 150° F.-155° F., 155° F.-160° F., 160° F.-165° F., 165° F.-170° F., 170° F.-175° F., 175° F.-180° F., 180° F.-185° F., 185° F.-190° F., 190° F.-195° F., or 195° F.-200° F. In one or more embodiments, third temperature may be between 175° F.-200° F. In some cases, third temperature may be approximately 150° F., approximately 155° F., approximately 160° F., approximately 165° F., approximately 170° F., approximately 175° F., approximately 180° F., approximately 185° F., approximately 190° F., approximately 195 OF, or approximately 200° F. In one or more embodiments, second time period may be between 2 hours and 4 hours, such as 2 hours-2.5 hours, 2.5 hours-3 hours, 3 hours-3.5 hours, or 3.5 hours-4 hours. In some cases, second time period may be approximately 2 hours, approximately 2.5 hours, approximately 3 hours, approximately 3.5 hours, or approximately 4 hours.

With continued reference to FIG. 3, at step 335, method 300 includes straining first raw material-food-grade oil mixture to separate first raw material from food-grade oil 136 and yield first extract 152. This step may be implemented with reference to details described above in this disclosure and without limitation.

With continued reference to FIG. 3, in one or more embodiments, method 300 may further include step 340. Step 340 includes mixing at least a portion of separated first raw material with additional food-grade oil 136 to form a recycled first raw material-food-grade oil mixture. In one or more embodiments, method 300 may further include step 345. Step 345 includes combining the recycled first raw material-food-grade oil mixture with first raw material-food-grade oil mixture. Such steps may facilitate secondary extraction of residue psilocybin-based compound 112 from first raw material and reduce waste.

With continued reference to FIG. 3, in one or more embodiments, method 300 may further include step 350. Step 350 includes separating an aqueous phase from an oil phase of first extract 152. In some cases, separating aqueous phase may include resting first extract 152 for one to three hours until the first extract 152 separates into an aqueous layer and an oil layer due to their incompatible polarities, and separating the aqueous layer from the oil layer. In some cases, separating aqueous layer may be performed by flowing liquid from a top or bottom of a stratified aqueous phase/oil phase mixture. In some cases, removal of aqueous phase may include removal of raw materials, aromatics, or other constituents used for extraction. In some cases, separating aqueous phase may include cooling first extract 152 to below 32° F., the freezing point of water, thereby freezing the aqueous phase into an ice layer, and separating the ice layer from a rest of the first extract 152.

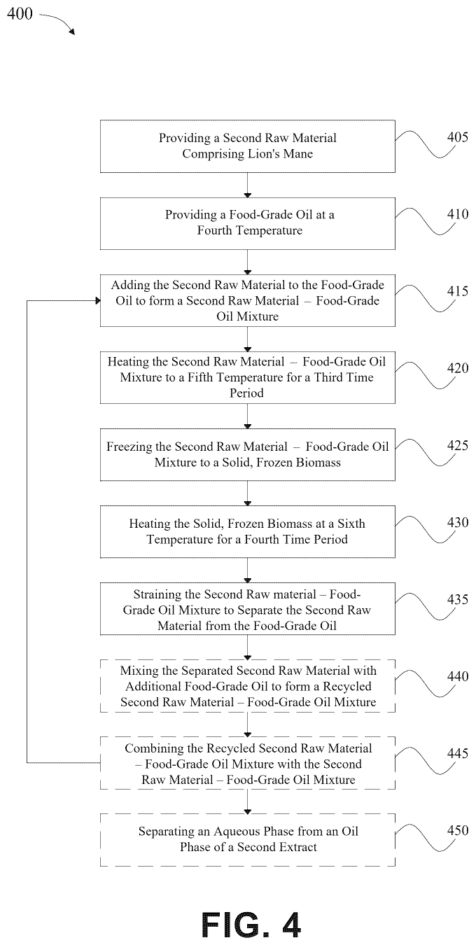

Referring now to FIG. 4, an exemplary embodiment of a method 400 for producing second extract 160 including second dosage 116 of lion's mane 120 is illustrated. Implementation of method 400 may be consistent with any details disclosed in U.S. patent application Ser. No. 17/949,126 (attorney docket number 1567-004USU1), filed on Sep. 20, 2022, entitled “MUSHROOM COMPOUNDS INFUSED CONSUMABLES AND NON-CONSUMABLES”, the entirety of which is incorporated herein by reference.

With continued reference to FIG. 4, at step 405, method 400 includes providing a second raw material including lion's mane 120. In one or more embodiments, second raw material may include a lion's mane mushroom, i.e., a mushroom of Hericium genus 164, consistent with details described above in this disclosure. In some cases, Hericium genus 164 may include species such as without limitation Hericium erinaceus, Hericium americanum, Hericium coralloides, Hericium abietis, Hericium alpestre, Hericium cirrhatum, Hericium rajendrae, and Hericium flagellum, among others.

With continued reference to FIG. 4, at step 410, method 400 includes providing food-grade oil 136 at a fourth temperature. This step may be implemented with reference to details described above in this disclosure and without limitation. In one or more embodiments, fourth temperature may be between 200° F.-240° F., such as 200° F.-210° F., 210° F.-220° F., 220° F.-230° F., 230° F.-240° F., 200° F.-205° F., 205° F.-210° F., 210° F.-215° F., 215° F.-220° F., 220° F.-225° F., 225° F.-230° F., 230° F.-235° F., or 235° F.-240° F. In some cases, fourth temperature may be approximately 200° F., approximately 205° F., approximately 210° F., approximately 215° F., approximately 220° F., approximately 225° F., approximately 230° F., approximately 235° F., or approximately 240° F.

With continued reference to FIG. 4, at step 415, method 400 includes adding at least a portion of second raw material to food-grade oil 136 to form a second raw material-food-grade oil mixture. This step may be implemented with reference to details described above in this disclosure and without limitation.

With continued reference to FIG. 4, at step 420, method 400 includes heating second raw material-food-grade oil mixture to a fifth temperature for a third time period. This step may be implemented with reference to details described above in this disclosure and without limitation. In one or more embodiments, fifth temperature may be between 200° F.-240° F., such as 200° F.-210° F., 210° F.-220° F., 220° F.-230° F., 230° F.-240° F., 200° F.-205° F., 205° F.-210° F., 210° F.-215° F., 215° F.-220° F., 220° F.-225° F., 225° F.-230° F., 230° F.-235° F., or 235° F.-240° F. In some cases, fifth temperature may be approximately 200° F., approximately 205° F., approximately 210° F., approximately 215° F., approximately 220° F., approximately 225° F., approximately 230° F., approximately 235° F., or approximately 240° F. In one or more embodiments, third time period may be between 4 hours and 9 hours, such as 4 hours-4.5 hours, 4.5 hours-5 hours, 5 hours-5.5 hours, 5.5 hours-6 hours, 6 hours-6.5 hours, 6.5 hours-7 hours, 7 hours-7.5 hours, 7.5 hours-8 hours, 8 hours-8.5 hours, or 8.5 hours-9 hours. In some cases, third time period may be approximately 4 hours, approximately 4.5 hours, approximately 5 hours, approximately 5.5 hours, approximately 6 hours, approximately 6.5 hours, approximately 7 hours, approximately 7.5 hours, approximately 8 hours, approximately 8.5 hours, or approximately 9 hours.

With continued reference to FIG. 4, at step 425, method 400 includes freezing second raw material-food-grade oil mixture into a solid, frozen biomass. This step may be implemented with reference to details described above in this disclosure and without limitation.

With continued reference to FIG. 4, at step 430, method 400 includes heating solid, frozen biomass at a sixth temperature for a fourth time period, thereby releasing lion's mane 120 from second raw material into food-grade oil 136. This step may be implemented with reference to details described above in this disclosure and without limitation. In one or more embodiments, sixth temperature may be between 150° F.-200° F., such as 150° F.-160° F., 160° F.-170° F., 170° F.-180° F., 180° F.-190° F., 190° F.-200° F., 150° F.-155° F., 155° F.-160° F., 160° F.-165° F., 165° F.-170° F., 170° F.-175° F., 175° F.-180° F., 180° F.-185° F., 185° F.-190° F., 190° F.-195° F., or 195° F.-200° F. In one or more embodiments, sixth temperature may be between 175° F.-200° F. In some cases, sixth temperature may be approximately 150° F., approximately 155° F., approximately 160° F., approximately 165° F., approximately 170° F., approximately 175° F., approximately 180° F., approximately 185° F., approximately 190° F., approximately 195° F., or approximately 200° F. In one or more embodiments, fourth time period may be between 2 hours and 4 hours, such as 2 hours-2.5 hours, 2.5 hours-3 hours, 3 hours-3.5 hours, or 3.5 hours-4 hours. In some cases, fourth time period may be approximately 2 hours, approximately 2.5 hours, approximately 3 hours, approximately 3.5 hours, or approximately 4 hours.

With continued reference to FIG. 4, at step 435, method 400 includes straining second raw material-food-grade oil mixture to separate second raw material from food-grade oil 136 and yield second extract 160. This step may be implemented with reference to details described above in this disclosure and without limitation.

With continued reference to FIG. 4, in one or more embodiments, method 400 may further include step 440. Step 440 includes mixing at least a portion of separated second raw material with additional food-grade oil 136 to form a recycled second raw material-food-grade oil mixture. In one or more embodiments, method 400 may further include step 445. Step 445 includes combining the recycled second raw material-food-grade oil mixture with second raw material-food-grade oil mixture. Such steps may facilitate secondary extraction of residue lion's mane 120 from second raw material and reduce waste.

With continued reference to FIG. 4, in one or more embodiments, method 400 may further include step 450. Step 450 includes separating an aqueous phase from an oil phase of first extract 152. In some cases, separating aqueous phase may include resting second extract 160 for one to three hours until the second extract 160 separates into an aqueous layer and an oil layer due to their incompatible polarities, and separating the aqueous layer from the oil layer. In some cases, separating aqueous phase may include cooling second extract 160 to below 32° F., the freezing point of water, thereby freezing the aqueous phase into an ice layer, and separating the ice layer from a rest of the second extract 160.

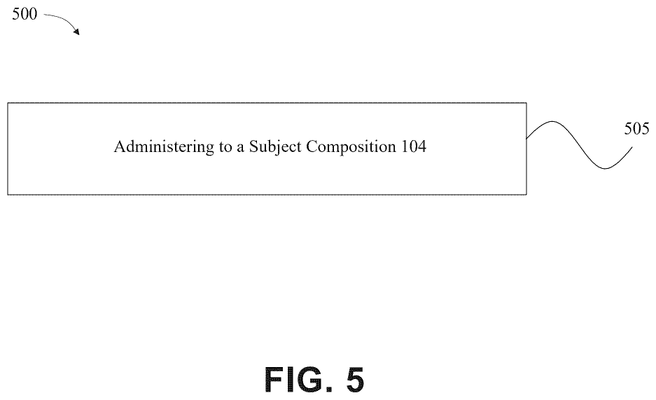

Referring now to FIG. 5, an exemplary embodiment of a method 500 for treating at least a medical condition is illustrated. Medical condition may include, without limitation, one or more of neurological condition, macular degeneration, retinal conditions, depression, concussion, pain including pain from neuroischemic disorder, anxiety, post-traumatic stress disorder (PTSD), cluster headaches, addiction, smoking cessation, obsessive compulsive disorder (OCD), attention deficit disorder (ADD), small blood vessel disease, Alzheimer's disease, dementia, Parkinson's disease, Huntington's disease, motor neuron disease, spinal muscular atrophy, stroke, spine injury, brain injury, peripheral neuropathy, fibromyalgia, migraine, menopause, weight loss, cancer, multiple sclerosis, hypoxia-ischemia injury, psychosis, nausea, inflammatory disease, rheumatoid arthritis, infection, inflammatory bowel disease, cardiovascular condition, and diabetic complication including diabetic polyneuropathy, among others, consistent with details described above in this disclosure.

With continued reference to FIG. 5, at step 505, method 500 includes administering, to subject, composition 104, consistent with details described above. Composition 104 includes a plurality of active ingredients, wherein the plurality of active ingredients includes a first dosage 108 of at least a psilocybin-based compound 112, a second dosage 116 of lion's mane 120, and a third dosage 124 of cilostazol 128. In one or more embodiments, administering composition 104 may include administering the composition 104 in a form of capsule 144. In one or more embodiments, administering composition 104 may include administering the composition 104 in a form of tablet 140. In one or more embodiments, administering composition 104 may include providing to subject an edible 148 including the composition 104. In one or more embodiments, administering composition 104 may include administering the composition 104 three times a week. In one or more embodiments, administering composition 104 may include administering the composition 104 once a week. In one or more embodiments, administering composition 104 may include administering the composition 104 on a daily basis. In one or more embodiments, administering composition 104 may include administering the composition 104 at a frequency between three times a day and once every two weeks.

With continued reference to FIG. 5, in some cases, method 500 may further include achieving within subject one or more benefits, such as without limitation, increasing neuroplasticity, accelerating growth of brain cells, and enhancing mental function, among others.

### Example

### Combination Therapy of Psilocybin, Lion's Mane, and Cilostazol: Pharmacokinetics, Pharmacodynamics, Mechanisms of Action, Safety, Dosing, and Application to Diabetic Neuropathy

We explore the potential synergistic effects of a combination therapy involving psilocybin, Lion's Mane (Hericium erinaceus), and cilostazol. Psilocybin, a serotonergic psychedelic, has shown promise in treating various psychiatric disorders. Lion's Mane, a medicinal mushroom, is known for its neurotrophic and cognitive-enhancing properties. Cilostazol, a phosphodiesterase-3 (PDE3) inhibitor, is used for its vasodilatory and anti-platelet effects. The pharmacokinetics, pharmacodynamics, safety, common dosing, and detailed mechanisms of action of these compounds are reviewed, with an emphasis on their combined therapeutic potential for diabetic neuropathy. Additionally, the epidemiology of diabetic neuropathy and relevant scientific literature are discussed.

### Introduction

The combination of psilocybin, Lion's Mane, and cilostazol presents an intriguing therapeutic approach for neuropsychiatric and neurodegenerative disorders, including diabetic neuropathy. Each component has distinct mechanisms of action that may provide complementary benefits. We review the current scientific literature on these compounds, focusing on their pharmacokinetics, pharmacodynamics, safety, common dosing, and potential synergistic effects in the context of diabetic neuropathy.

### Epidemiology of Diabetic Neuropathy

Diabetic neuropathy is one of the most common complications of diabetes mellitus, affecting approximately 50% of individuals with diabetes during their lifetime (Tesfaye et al., 2011). It encompasses a spectrum of nerve disorders caused by diabetes, with the most prevalent form being distal symmetric polyneuropathy (DSPN) (Feldman et al., 2019). DSPN primarily affects the peripheral nerves, leading to symptoms such as pain, numbness, and tingling in the extremities (Pop-Busui et al., 2017). The prevalence of diabetic neuropathy increases with the duration of diabetes, poor glycemic control, and the presence of other comorbid conditions such as hypertension and dyslipidemia. Diabetic polyneuropathy is the most common type of diabetic neuropathy, affecting multiple peripheral nerves throughout the body. The mechanisms underlying this condition involve a complex interplay of metabolic, vascular, and inflammatory factors that lead to nerve damage.

### Key Mechanisms Affecting Peripheral Nerve Cells in Diabetic PolyNeuropathy:

### 1. Hyperglycemia-Induced Metabolic Disturbances:

Polyol Pathway Overactivity: In hyperglycemia, glucose is shunted into the polyol pathway where it is reduced to sorbitol by aldose reductase. Accumulation of sorbitol and fructose within nerve cells may lead to osmotic stress and reduced levels of myo-inositol, impairing nerve function and conduction.

Formation of Advanced Glycation End Products (AGEs): High glucose levels result in non-enzymatic glycation of proteins and lipids, forming AGEs. AGEs may alter the structure and function of cellular proteins, contributing to neuronal damage and dysfunction.

### 2. Oxidative Stress:

Reactive Oxygen Species (ROS) Production: Hyperglycemia increases the production of ROS, overwhelming the antioxidant defense mechanisms of nerve cells. ROS may cause direct damage to DNA, proteins, and lipids, leading to cellular dysfunction and apoptosis.

Mitochondrial Dysfunction: Hyperglycemia-induced ROS may impair mitochondrial function, leading to energy deficits in nerve cells and contributing to axonal degeneration and cell death.

### 3. Protein Kinase C (PKC) Activation:

Hyperglycemia activates PKC, which affects various cellular processes, including blood flow regulation, vascular permeability, and inflammation. PKC activation may lead to reduced blood flow and increased vascular permeability, contributing to nerve ischemia and damage.

### 4. Inflammatory Pathways:

Pro-inflammatory Cytokines: Chronic hyperglycemia induces the production of pro-inflammatory cytokines such as TNF-α, IL-1β, and IL-6. These cytokines may activate inflammatory pathways, causing further damage to nerve cells and supporting cells (e.g., Schwann cells).

Nuclear Factor-kappa B (NF-κB) Activation: Inflammatory cytokines may activate NF-κB, a transcription factor that promotes the expression of genes involved in inflammation and cell survival. Persistent activation of NF-κB may contribute to chronic inflammation and neuronal damage.

### 5. Vascular Insufficiency:

Endothelial Dysfunction: Hyperglycemia may impair endothelial function, reducing the production of nitric oxide (NO) and causing vasoconstriction. This may lead to decreased blood supply (ischemia) to peripheral nerves, resulting in hypoxia and nutrient deficiency.

Microvascular Damage: Damage to the small blood vessels (vasa nervorum) that supply peripheral nerves may reduce perfusion and oxygenation, exacerbating nerve damage.

### 6. Impaired Neurotrophic Support:

Reduced Nerve Growth Factor (NGF) Levels: Hyperglycemia may decrease the production and availability of neurotrophic factors like NGF, which are essential for the growth, maintenance, and survival of neurons. Reduced NGF levels may contribute to neuronal atrophy and apoptosis.

### Pharmacokinetics and Pharmacodynamics

### Psilocybin

Pharmacokinetics: Psilocybin, the active compound in Psilocybe mushrooms, is rapidly converted to its active metabolite, psilocin, in the body. Psilocin is primarily responsible for the psychedelic effects. Psilocin is absorbed in the gastrointestinal tract, with peak plasma concentrations occurring within 90-120 minutes post-ingestion. It has a half-life of approximately 2-4 hours and is primarily metabolized by the liver through glucuronidation and sulfation, with excretion mainly via the kidneys.

Pharmacodynamics: Psilocybin may exert its effects primarily through agonism at the 5-HT2A receptor, a subtype of the serotonin receptor (Nichols, 2004). This receptor is widely distributed in the brain, particularly in areas involved in cognition and perception (Vollenweider & Kometer, 2010). Activation of the 5-HT2A receptor leads to increased glutamate release in the prefrontal cortex, enhancing synaptic plasticity and connectivity. Psilocybin may also affect other serotonin receptors, including 5-HT1A and 5-HT2C, contributing to its complex psychotropic profile (Nichols, 2004).

Lion's Mane (Hericium erinaceus)

Pharmacokinetics: The bioactive compounds in Lion's Mane, including hericenones and erinacines, may be absorbed in the gastrointestinal tract. However, detailed pharmacokinetic data on these compounds are limited. Preliminary studies suggest that they cross the blood-brain barrier and are distributed to the central nervous system.

Pharmacodynamics: Lion's Mane is known for its neurotrophic and neuroprotective effects. The hericenones and erinacines may stimulate the production of nerve growth factor (NGF) and brain-derived neurotrophic factor (BDNF), which are critical for the growth, maintenance, and survival of neurons. Additionally, Lion's Mane may have anti-inflammatory and antioxidant properties, which contribute to its neuroprotective effects.

Lion's mane (Hericium erinaceus) is a medicinal mushroom that has garnered attention for its potential therapeutic benefits, including its effects on diabetic neuropathy. Diabetic neuropathy is a type of nerve damage that can occur with diabetes, leading to symptoms like pain, numbness, and weakness, particularly in the hands and feet. Research suggests that lion's mane may offer several benefits for this condition:
1. Neuroprotective Effects: Lion's mane contains bioactive compounds such as hericenones and erinacines that may promote nerve growth factor (NGF) synthesis. NGF plays a crucial role in the growth, maintenance, and survival of neurons. This may help repair damaged nerves and potentially alleviate symptoms of diabetic neuropathy.
2. Anti-inflammatory Properties: Chronic inflammation is a significant contributor to diabetic neuropathy. Lion's mane has been shown to possess anti-inflammatory properties, which may help reduce inflammation in the nervous system and improve nerve function.
3. Antioxidant Activity: Oxidative stress is another factor implicated in diabetic neuropathy. Lion's mane has potent antioxidant effects, which can help mitigate oxidative damage to neurons, thus preserving nerve function.
4. Regeneration of Nerve Cells: Studies indicate that lion's mane may enhance the regeneration of nerve cells. This is particularly beneficial in diabetic neuropathy, where nerve damage is a common issue.

### Cilostazol

Pharmacokinetics: Cilostazol may be well-absorbed orally, with peak plasma concentrations achieved within 2-3 hours. It has a half-life of approximately 11-13 hours and is metabolized by the liver via cytochrome P450 enzymes, primarily CYP3A4 and CYP2C19. Cilostazol and its active metabolites may be excreted in urine and feces.

Pharmacodynamics: Cilostazol may inhibit phosphodiesterase-3 (PDE3), leading to an increase in intracellular cyclic adenosine monophosphate (cAMP). Elevated cAMP levels result in vasodilation and inhibition of platelet aggregation. Cilostazol may also have neuroprotective effects, as it enhances cerebral blood flow and exerts anti-inflammatory actions in the brain.

Cilostazol is a phosphodiesterase III inhibitor primarily used to treat intermittent claudication in peripheral arterial disease. However, emerging evidence suggests that cilostazol may also have beneficial effects on diabetic neuropathy due to its vasodilatory, antiplatelet, and neuroprotective properties. Here are some of the key benefits:
1. Improved Blood Flow: Cilostazol promotes vasodilation and improves microcirculation, which may enhance blood flow to peripheral nerves. This improved perfusion can help alleviate symptoms of diabetic neuropathy.
2. Neuroprotective Effects: Cilostazol has been shown to exert neuroprotective effects, which may help protect neurons from damage caused by hyperglycemia and other metabolic disturbances in diabetes.
3. Anti-inflammatory Properties: By inhibiting phosphodiesterase III, cilostazol may reduce the production of inflammatory cytokines. This may decrease inflammation in peripheral nerves, potentially reducing pain and other symptoms of diabetic neuropathy.
4. Antioxidant Activity: Cilostazol may enhance the expression of antioxidant enzymes and reduce oxidative stress, which is a significant contributor to nerve damage in diabetic neuropathy.
5. Improved Nerve Function: Clinical studies suggest that cilostazol can improve nerve conduction velocity and reduce the symptoms of diabetic neuropathy, such as numbness,

### Mechanisms of Action

### Mechanisms of Psilocybin in Diabetic Neuropathy

### 1. Modulation of Serotonin Receptors

Psilocybin's primary mechanism of action may involve its agonistic activity at the 5-HT2A receptor. Activation of this receptor may lead to various downstream effects that may be beneficial in treating diabetic neuropathy:

Pain Modulation: Activation of the 5-HT2A receptor has been shown to modulate pain perception. Psilocybin may reduce neuropathic pain by altering the central processing of pain signals, potentially providing significant relief for patients with diabetic neuropathy.

Anti-inflammatory Effects: Serotonin receptors, including 5-HT2A, are involved in modulating inflammation. Psilocybin's activation of these receptors may lead to a reduction in pro-inflammatory cytokines and mediators, thus reducing neuroinflammation, a key contributor to neuropathic pain and nerve damage in diabetic neuropathy.

### 2. Neuroplasticity and Neurogenesis

Psilocybin has been shown to promote neuroplasticity, the brain's ability to reorganize itself by forming new neural connections. This effect is crucial for repairing damaged nerves and restoring function in diabetic neuropathy:

Synaptogenesis: Studies have demonstrated that psilocybin may induce synaptogenesis, the formation of new synapses between neurons. This process may help repair damaged neural circuits and enhance neural connectivity, which is often impaired in diabetic neuropathy.

BDNF Upregulation: Brain-derived neurotrophic factor (BDNF) is essential for the growth and maintenance of neurons. Psilocybin has been shown to increase BDNF levels, promoting neuronal survival and regeneration, which could be particularly beneficial for nerve repair in diabetic neuropathy.

### 3. Enhanced Neuroprotection

Psilocybin's neuroprotective effects could also play a significant role in managing diabetic neuropathy:

Antioxidant Activity: Psilocybin has been reported to exhibit antioxidant properties, which may help protect neurons from oxidative stress, a major factor in diabetic neuropathy progression.

Mitochondrial Function: Improved mitochondrial function and energy production in neurons can enhance their resilience against diabetic neuropathy-induced damage.

Psilocybin's effects on cellular metabolism may contribute to this protective mechanism.

### Lion's Mane and Neurotrophic Support

### Mechanism of Action

The potential mechanisms by which lion's mane exerts its beneficial effects on diabetic neuropathy may include:

Stimulation of NGF Synthesis: The hericenones and erinacines in lion's mane may stimulate the production of NGF, which supports the growth and survival of neurons.

Reduction of Inflammatory Cytokines: Lion's mane may inhibit the production of proinflammatory cytokines, thereby reducing inflammation and associated nerve damage.

Scavenging of Free Radicals: The antioxidant properties of lion's mane help neutralize free radicals, reducing oxidative stress and preventing further neuronal damage.

Enhancement of Myelination: Some studies suggest that lion's mane can promote the myelination of nerve fibers, which is essential for proper nerve signal transmission.

Lion's Mane's ability to stimulate NGF and BDNF production may support neurogenesis and synaptic plasticity. This may be particularly relevant for neurodegenerative conditions, where the maintenance and growth of neurons are compromised. The antioxidant and anti-inflammatory properties of Lion's Mane may further enhance its neuroprotective potential.

### Cilostazol and Neuroprotection

### Mechanism of Action

The beneficial effects of cilostazol on diabetic neuropathy may be mediated through several mechanisms:

Inhibition of Phosphodiesterase III: Cilostazol may inhibit phosphodiesterase III, leading to an increase in cyclic adenosine monophosphate (cAMP) levels. Elevated cAMP may promote vasodilation and improve blood flow, particularly in microvascular beds.

Reduction of Inflammatory Cytokines: Cilostazol may inhibit the release of proinflammatory cytokines such as TNF-α and IL-1β. This reduction in inflammation may prevent or mitigate nerve damage associated with diabetic neuropathy.

Antioxidant Effects: Cilostazol may upregulate the expression of antioxidant enzymes like superoxide dismutase (SOD) and glutathione peroxidase (GPx). This may enhances the body's ability to neutralize oxidative stress, thereby protecting peripheral nerves from damage.

Neuroprotective Actions: By increasing CAMP, cilostazol may exert neuroprotective effects that promote neuronal survival and repair, aiding in the maintenance of nerve function.

Cilostazol's inhibition of PDE3 may lead to increased cAMP levels, which promote vasodilation and improve cerebral blood flow. This may be crucial in conditions characterized by reduced brain perfusion, such as stroke and vascular dementia. The anti-inflammatory effects of cilostazol may add another layer of neuroprotection, potentially mitigating the effects of chronic neuroinflammation.

### Safety and Common Dosing

### Psilocybin

Safety: Psilocybin is generally considered safe when used in controlled settings, though it may cause transient increases in heart rate and blood pressure, nausea, and psychological distress. It is contraindicated in individuals with a history of psychotic disorders or severe cardiovascular conditions.

Common Dosing: Typical doses of psilocybin range from 10 to 30 mg, with lower doses (e.g., 10-15 mg) often used for mild to moderate therapeutic effects, and higher doses (20-30 mg) for more profound psychotropic experiences. The nano-therapeutic dosing regimen (0.5-1.0 mg) per day should be tailored to individual patient needs to be determined by clinical trials.

Lion's Mane (Hericium erinaceus)

Safety: Lion's Mane is considered safe for most people, with rare reports of mild gastrointestinal discomfort or allergic reactions. It has a long history of use in traditional medicine with no significant adverse effects reported.

Common Dosing: The typical dose of Lion's Mane ranges from 500 mg to 3,000 mg per day, usually taken in divided doses. Standardized extracts containing high concentrations of hericenones and erinacines are often used to ensure consistency and potency. A dose of 500 mg-3000 mg per day will be evaluated in clinical trials for clinical efficacy.

### Cilostazol

Safety: Cilostazol is generally well-tolerated but may cause side effects such as headache, diarrhea, and palpitations. It is contraindicated in patients with heart failure due to its potential to exacerbate the condition. Regular monitoring is recommended for patients with cardiovascular conditions.

Common Dosing: The usual dose of cilostazol is 100 mg taken twice daily. Dose adjustments may be necessary based on individual tolerance and therapeutic response.

### Application to Diabetic Neuropathy

Diabetic neuropathy is a debilitating condition characterized by pain, sensory loss, and motor dysfunction (Feldman et al., 2019). Current treatments are often inadequate, necessitating the exploration of novel therapeutic approaches. The combination of psilocybin, Lion's Mane, and cilostazol may provide a comprehensive treatment strategy addressing multiple pathophysiological aspects of diabetic neuropathy.

Psilocybin: By enhancing neuroplasticity and modulating serotoninergic pathways, psilocybin may alleviate neuropathic pain and improve mood and cognitive function in diabetic neuropathy patients (Carhart-Harris et al., 2017).

Lion's Mane: Its neurotrophic effects may promote nerve regeneration and repair, while its anti-inflammatory properties may reduce nerve inflammation and associated pain.

Cilostazol: By improving blood flow and providing neuroprotection, cilostazol could mitigate the vascular contributions to diabetic neuropathy and support overall nerve health.

### Clinical Implications

While the potential mechanisms of psilocybin in treating diabetic neuropathy are promising, clinical studies are needed to confirm its efficacy and safety in this context. Preliminary evidence suggests that psilocybin may provide significant pain relief, promote nerve regeneration, and protect against further nerve damage. These effects may improve the quality of life for patients suffering from diabetic neuropathy, offering a novel approach where traditional therapies have failed.

### CONCLUSION

Psilocybin-Lion's Mane-Cilostazol combination holds significant potential as a therapeutic agent for diabetic neuropathy due to its ability to modulate pain, reduce inflammation, promote neuroplasticity and neurogenesis, and protect neurons from damage. Further research, including clinical trials, is essential to fully understand its benefits and risks in treating this debilitating condition. If proven effective, this combination could become a valuable addition to the treatment options for diabetic neuropathy, addressing both the symptoms and underlying nerve damage.

### SCIENTIFIC LITERATURE

1. Vollenweider, F. X., & Kometer, M. (2010). The neurobiology of psychedelic drugs: Implications for the treatment of mood disorders. Nature Reviews Neuroscience, 11 (9), 642-651.
2. Nichols, D. E. (2004). Hallucinogens. Pharmacology & Therapeutics, 101 (2), 131-181.
3. Flanagan, T. W., & Nichols, C. D. (2018). Psychedelics as anti-inflammatory agents. International Review of Psychiatry, 30 (4), 363-375.
4. Szabo, A., Kovacs, A., Frecska, E., & Rajnavolgyi, E. (2014). Psychedelic N,Ndimethyltryptamine and 5-methoxy-N,N-dimethyltryptamine modulate innate and adaptive inflammatory responses through the sigma-1 receptor of human monocyte derived dendritic cells. PLOS ONE, 9 (8), e106533.
5. Ly, C., et al. (2018). Psychedelics promote structural and functional neural plasticity. Cell Reports, 23 (11), 3170-3182.
6. Carhart-Harris, R. L., et al. (2017). Psilocybin with psychological support for treatment-resistant depression: six-month follow-up. Psychopharmacology, 235 (2), 399-408.
7. Catlow, B. J., Song, S., Paredes, D. A., Kirstein, C. L., & Sanchez-Ramos, J. (2013). Effects of psilocybin on hippocampal neurogenesis and extinction of trace fear conditioning. Experimental Brain Research, 228 (4), 481-491.
8. Nutt, D., King, L. A., & Nichols, D. E. (2013). Effects of Schedule I drug laws on neuroscience research and treatment innovation. Nature Reviews Neuroscience, 14 (8), 577-585.
9. Feldman E L, Nave K A, Jensen T S, Bennett D L H. “New Horizons in Diabetic Neuropathy: Mechanisms, Bioenergetics, and Pain.” Neuron. This paper explores the latest insights into the mechanisms of diabetic neuropathy, including metabolic, bioenergetic, and pain pathways.
10. Vincent A M, Callaghan B C, Smith A L, Feldman E L. “Diabetic Neuropathy: Cellular Mechanisms as Therapeutic Targets.” Nature Reviews Neurology. This review discusses cellular mechanisms underlying diabetic neuropathy and potential therapeutic targets.
11. Pop-Busui R, Ang L, Holmes C, Gallagher K, Feldman E L. “Mechanisms of Disease: Diabetic Neuropathies-Mechanisms, Emerging Treatments, and Subtypes.” Nature Reviews Endocrinology. This article provides an overview of the mechanisms, emerging treatments, and subtypes of diabetic neuropathies.
12. Srinivasan S, Stevens M, Wiley J W. “Diabetic Peripheral Neuropathy: Evidence for Apoptosis and Associated Mitochondrial Dysfunction.” Diabetes. This paper provides evidence for the role of apoptosis and mitochondrial dysfunction in diabetic peripheral neuropathy.
13. Feldman, E. L., et al. (2019). Diabetic neuropathy. Nature Reviews Disease Primers, 5 (1), 41. This comprehensive review provides detailed information on the prevalence, pathophysiology, and clinical features of diabetic neuropathy.
14. Pop-Busui, R., et al. (2017). Diabetic neuropathy: A position statement by the American Diabetes Association. Diabetes Care, 40 (1), 136-154. This position statement outlines the current understanding of diabetic neuropathy and provides guidelines for its diagnosis and management.
15. Tesfaye, S., et al. (2011). Diabetic neuropathies: Update on definitions, diagnostic criteria, estimation of severity, and treatments. Diabetes Care, 33 (10), 2285-2293. This paper provides updated definitions and diagnostic criteria for diabetic neuropathies, as well as a review of available treatments.

The foregoing has been a detailed description of illustrative embodiments of the invention. Various modifications and additions can be made without departing from the spirit and scope of this invention. Features of each of the various embodiments described above may be combined with features of other described embodiments as appropriate in order to provide a multiplicity of feature combinations in associated new embodiments. Furthermore, while the foregoing describes a number of separate embodiments, what has been described herein is merely illustrative of the application of the principles of the present invention. Additionally, although particular methods herein may be illustrated and/or described as being performed in a specific order, the ordering is highly variable within ordinary skill to achieve methods, systems, and software according to the present disclosure. Accordingly, this description is meant to be taken only by way of example, and not to otherwise limit the scope of this invention.

In some embodiments, a pharmaceutical composition may include one or more components at a dose or within a dose range as described in Table 1.

TABLE 1

| | | |
|---|---|---|
| Minimum | Nominal | Maximum |

| | | | | | |
|---|---|---|---|---|---|
| Psilocybin | 0.01 | mg | 0.1 mg-10 mg | 10 | mg |
| Lion's mane | 5 | mg | 100 mg-1000 mg | 1500 | mg |
| Cilostazol | 2.5 | mg | 25 mg-200 mg | 200 | mg |

In some embodiments, a pharmaceutical composition may include one or more ingredients and/or forms described in Table 2.

TABLE 2

| | |
|---|---|
| Form | Capsule, tablet, edible, gel, powder |
| Additive | Vitamin C, vitamin A, zinc, antioxidant |
| Matrix | Food-grade oil, lactose, microcrystalline cellulose, starch, calcium phosphate, |
| | calcium carbonate, sucrose, maltodextrin, mannitol, sorbitol, and sodium |
| | chloride |
| Food-grade | Olive oil (Olea europaea), canola oil (Brassica napus), sunflower oil (Helianthus |
| oil | annuus), soybean oil (Glycine max), peanut oil (Arachis hypogaea), sesame oil |
| | (Sesamum indicum), corn oil (Zea mays), coconut oil (Cocos nucifera), palm oil |
| | (Elaeis guineensis), palm kernel oil (Elaeis guineensis), avocado oil (Persea |
| | americana), grapeseed oil (Vitis vinifera), walnut oil (Juglans regia), hazelnut oil |
| | (Corylus avellana), almond oil (Prunus dulcis), macadamia nut oil (Macadamia |
| | integrifolia), pistachio oil (Pistacia vera), flaxseed oil (Linum usitatissimum), |
| | hemp seed oil (Cannabis sativa), pumpkin seed oil (Cucurbita pepo), rice bran |
| | oil (Oryza sativa), safflower oil (Carthamus tinctorius), mustard oil (Brassica |
| | juncea), camelina oil (Camelina sativa), cottonseed oil (Gossypium spp.), chia |
| | seed oil (Salvia hispanica), perilla oil (Perilla frutescens), moringa oil (Moringa |
| | oleifera), argan oil (Argania spinosa), pecan oil (Carya illinoinensis), medium |
| | chain triglycerides oil, vegetable oil, fish oil, butter, shortening, margarine, and |
| | lard |
| Excipient | binders, fillers/diluents, disintegrants, lubricants, coatings, preservatives, |
| | stabilizers, acidifying agents (acetic acid, glacial acetic acid, citric acid, fumaric |
| | acid, hydrochloric acid, diluted hydrochloric acid, malic acid, nitric acid, |
| | phosphoric acid, diluted phosphoric acid, sulfuric acid, and tartaric acid, etc.); |
| | alkalizing agents (ammonia solution, ammonium carbonate, diethanolamine, |
| | diisopropanolamine, potassium hydroxide, sodium bicarbonate, sodium borate, |
| | sodium carbonate, sodium hydroxide, and trolamine, etc.); antifoaming agents |
| | (dimethicone, simethicone, and/or the like); antimicrobial preservatives |
| | (benzalkonium chloride, benzalkonium chloride solution, benzethonium |
| | chloride, benzoic acid, benzyl alcohol, butylparaben, cetylpyridinium chloride, |
| | chlorobutanol, chlorocresol, cresol, dehydroacetic acid, ethylparaben, |
| | methylparaben, methylparaben sodium, phenol, phenylethyl alcohol, |
| | phenylmercuric acetate, phenylmercuric nitrate, potassium benzoate, potassium |
| | sorbate, propylparaben, propylparaben sodium, sodium benzoate, sodium |
| | dehydroacetate, sodium propionate, ascorbic acid, thimerosal, thymol, and/or the |
| | like); antioxidants (ascorbic acid, ascorbyl palmitate, butylated hydroxyanisole, |
| | butylated hydroxytoluene, hypophosphorous acid, monothioglycerol, propyl |
| | gallate, sodium formaldehyde sulfoxylate, sodium metabisulfite, sodium |
| | thiosulfate, sulfur dioxide, tocopherol, tocopherols excipient, and/or the like); |
| | buffering agents (acetic acid, ammonium carbonate, ammonium phosphate, boric |
| | acid, citric acid, lactic acid, phosphoric acid, potassium citrate, potassium |
| | metaphosphate, potassium phosphate monobasic, sodium acetate, sodium citrate, |
| | sodium lactate solution, dibasic sodium phosphate, monobasic sodium |
| | phosphate, and/or the like); chelating agents (edetate disodium, |
| | ethylenediaminetetraacetic acid and salts, edetic acid, and/or the like); coating |
| | agents (sodium carboxymethylcellulose, cellulose acetate, cellulose acetate |
| | phthalate, ethylcellulose, gelatin, pharmaceutical glaze, hydroxypropyl cellulose, |
| | hydroxypropyl methylcellulose, hydroxypropyl methylcellulose phthalate, |
| | methacrylic acid copolymer, methylcellulose, polyvinyl acetate phthalate, |
| | shellac, sucrose, titanium dioxide, carnauba wax, microcrystalline wax, zein, |
| | and/or the like); colorants (caramel, red, yellow, black or blends, ferric oxide, |
| | and/or the like); complexing agents (ethylenediaminetetraacetic acid (EDTA) |
| | and salts thereof, edetic acid, gentisic acid ethanolamide, oxyquinoline sulfate, |
| | and/or the like); desiccants (calcium chloride, calcium sulfate, silicon dioxide, |
| | and/or the like); emulsifying and/or solubilizing agents (acacia, cholesterol, |
| | diethanolamine (adjunct), glyceryl monostearate, lanolin alcohols, mono- and di- |
| | glycerides, monoethanolamine (adjunct), lecithin, oleic acid (adjunct), oleyl |
| | alcohol (stabilizer), poloxamer, polyoxyethylene 50 stearate, polyoxyl 35 castor |
| | oil, polyoxyl 40 hydrogenated castor oil, polyoxyl 10 oleyl ether, polyoxyl 20 |
| | cetostearyl ether, polyoxyl 40 stearate, polysorbate 20, polysorbate 40, |
| | polysorbate 60, polysorbate 80, diacetate, monostearate, sodium lauryl sulfate, |
| | sodium stearate, sorbitan monolaurate, sorbitan monooleate, sorbitan |
| | monopalmitate, sorbitan monostearate, stearic acid, trolamine, emulsifying wax, |
| | and/or the like); filtering aids (powdered cellulose, purified siliceous earth, |
| | and/or the like); flavors and perfumes (anethole, benzaldehyde, ethyl vanillin, |
| | menthol, methyl salicylate, monosodium glutamate, orange flower oil, |
| | peppermint, peppermint oil, peppermint spirit, rose oil, stronger rose water, |
| | thymol, tolu balsam tincture, vanilla, vanilla tincture, vanillin, and/or the like); |
| | humectants (glycerol, hexylene glycol, sorbitol, and/or the like); plasticizers |
| | (castor oil, diacetylated monoglycerides, diethyl phthalate, glycerol, mono- and |
| | di-acetylated monoglycerides, propylene glycol, triacetin, triethyl citrate, and/or |
| | the like); polymers (cellulose acetate, alkyl celluloses, hydroxyalkyl, acrylic |
| | polymers and copolymers, and/or the like); solvents (acetone, alcohol, diluted |
| | alcohol, amylene hydrate, benzyl benzoate, butyl alcohol, carbon tetrachloride, |
| | chloroform, corn oil, cottonseed oil, ethyl acetate, glycerol, hexylene glycol, |
| | isopropyl alcohol, methyl alcohol, methylene chloride, methyl isobutyl ketone, |
| | mineral oil, peanut oil, propylene carbonate, sesame oil, water for injection, |
| | sterile water for injection, sterile water for irrigation, purified water, and/or the |
| | like); sorbents (powdered cellulose, charcoal, purified siliceous earth, and/or the |
| | like); carbon dioxide sorbents (barium hydroxide lime, soda lime, and/or the |
| | like); stiffening agents (hydrogenated castor oil, cetostearyl alcohol, cetyl |
| | alcohol, cetyl esters wax, hard fat, paraffin, polyethylene excipient, stearyl |
| | alcohol, emulsifying wax, white wax, yellow wax, and/or the like); suspending |
| | and/or viscosity-increasing agents (acacia, agar, alginic acid, aluminum |
| | monostearate, bentonite, purified bentonite, magma bentonite, carbomer, |
| | carboxymethylcellulose calcium, carboxymethylcellulose sodium, |
| | carboxymethylcellulose sodium carrageenan, microcrystalline and |
| | carboxymethylcellulose sodium cellulose, dextrin, gelatin, guar gum, |
| | hydroxyethyl cellulose, hydroxypropyl cellulose, hydroxypropyl |
| | methylcellulose, magnesium aluminum silicate, methylcellulose, pectin, |
| | polyethylene oxide, polyvinyl alcohol, povidone, alginate, silicon dioxide, |
| | colloidal silicon dioxide, sodium alginate, tragacanth, xanthan gum, and/or the |
| | like); sweetening agents (aspartame, dextrates, dextrose, excipient dextrose, |
| | fructose, mannitol, saccharin, calcium saccharin, sodium saccharin, sorbitol, |
| | solution sorbitol, sucrose, compressible sugar, confectioner's sugar, syrup, |
| | and/or the like); surfactants (simethicone, etc.); tablet binders (acacia, alginic |
| | acid, sodium carboxymethylcellulose, microcrystalline cellulose, dextrin, |
| | ethylcellulose, gelatin, liquid glucose, guar gum, hydroxypropyl methylcellulose, |
| | methylcellulose, polyethylene oxide, povidone, pregelatinized starch, syrup, |
| | and/or the like); tablet and/or capsule diluents (calcium carbonate, dibasic |
| | calcium phosphate, tribasic calcium phosphate, calcium sulfate, microcrystalline |
| | cellulose, powdered cellulose, dextrates, dextrin, dextrose excipient, fructose, |
| | kaolin, lactose, mannitol, sorbitol, starch, pregelatinized starch, sucrose, |
| | compressible sugar, confectioner's sugar, and/or the like); tablet disintegrants |
| | (alginic acid, microcrystalline cellulose, croscarmellose sodium, crospovidone, |
| | polacrilin potassium, sodium starch glycolate, starch, pregelatinized starch, |
| | and/or the like); tablet and/or capsule lubricants (calcium stearate, glyceryl |
| | behenate, magnesium stearate, light mineral oil, sodium stearyl fumarate, stearic |
| | acid, purified stearic acid, talc, hydrogenated vegetable oil, zinc stearate, and/or |
| | the like); thickening agents (gelatin having a bloom strength of 50-100, and/or |
| | the like); tonicity agent (dextrose, glycerol, mannitol, potassium chloride, |
| | sodium chloride, and/or the like); flavored and/or sweetened vehicle (aromatic |
| | elixir, compound benzaldehyde elixir, iso-alcoholic elixir, peppermint water, |
| | sorbitol solution, syrup, tolu balsam syrup, and/or the like); oleaginous vehicle |
| | (almond oil, corn oil, cottonseed oil, ethyl oleate, isopropyl myristate, isopropyl |
| | palmitate, mineral oil, light mineral oil, myristyl alcohol, octyl dodecanol, olive |
| | oil, peanut oil, persic oil, sesame oil, soybean oil, squalane, and/or the like); |
| | vehicle: solid carrier (sugar spheres, etc.); sterile vehicle (bacteriostatic water for |
| | injection, bacteriostatic sodium chloride injection, and/or the like); viscosity- |
| | increasing agents (see suspending agent above); water-repelling agents |
| | (cyclomethicone, dimethicone, simethiconevehicle, and/or the like); and/or |
| | solubilizing agent (benzalkonium chloride, benzethonium chloride, |
| | cetylpyridinium chloride, docusate sodium, nonoxynol 9, nonoxynol 10, |
| | octoxynol 9, poloxamer, polyoxyl 35 castor oil, polyoxyl 40, hydrogenated |
| | castor oil, polyoxyl 50 stearate, polyoxyl 10 oleyl ether, polyoxyl 20, cetostearyl |
| | ether, polyoxyl 40 stearate, polysorbate 20, polysorbate 40, polysorbate 60, |
| | polysorbate 80, sodium lauryl sulfate, sorbitan monolaurate, sorbitan |
| | monooleate, sorbitan monopalmitate, sorbitan monostearate, tyloxapol) |

The following is a list of embodiments contemplated by the present disclosure:

A1. A composition of matter for treating at least a medical condition pertaining to a subject; the composition comprising:
a plurality of active ingredients, wherein the plurality of active ingredients comprises:
a first dosage of at least a psilocybin-based compound;
a second dosage of lion's mane; and
a third dosage of cilostazol.

A2. The composition of embodiment A1, wherein the at least a psilocybin-based compound comprises psilocybin or psilocin.

A3. The composition of embodiment A1, wherein the first dosage of at least a psilocybin-based compound is between 0.05 mg and 10 mg.

A4. The composition of embodiment A3, wherein the first dosage of at least a psilocybin-based compound is approximately 0.5 mg.

A5. The composition of embodiment A1, wherein the composition comprises between 0.1% and 99.7% by weight of the first dosage of at least a psilocybin-based compound.

A6. The composition of embodiment A1, wherein the second dosage of lion's mane is between 5 mg and 1500 mg.

A7. The composition of embodiment A6, wherein the second dosage of lion's mane is approximately 250 mg.

A8. The composition of embodiment A1, wherein the composition comprises between 0.1% and 99.7% by weight of the second dosage of lion's mane.

A9. The composition of embodiment A1, wherein the third dosage of cilostazol is between 25 mg and 100 mg.

A10. The composition of embodiment A9, wherein the third dosage of cilostazol is approximately 50 mg.

A11. The composition of embodiment A1, wherein the composition comprises between 0.1% and 99.7% by weight of the third dosage of cilostazol.

A12. The composition of embodiment A1, further comprising a matrix, wherein the composition comprises between 0.1% and 99.7% by weight of the matrix.

A13. The composition of embodiment A12, wherein the matrix includes one or more members selected from a group consisting of food-grade oil, lactose, microcrystalline cellulose, starch, calcium phosphate, calcium carbonate, sucrose, maltodextrin, mannitol, sorbitol, and sodium chloride.

A14. The composition of embodiment A1, wherein the composition is in one or more of a tablet or a capsule form.

A15. The composition of embodiment A1, wherein the composition is in an edible form.

A16. The composition of embodiment A1, wherein the composition comprises at least a food-grade oil.

A17. The composition of embodiment A16, wherein one or more of the first dosage of at least a psilocybin-based compound and the second dosage of lion's mane is dissolved in the at least a food-grade oil.

A18. The composition of embodiment A17, wherein the third dosage of cilostazol is dissolved in the at least a food-grade oil.

A19. The composition of embodiment A16, wherein the at least a food-grade oil comprises one or more members selected from a group consisting of olive oil (Olea europaea), canola oil (Brassica napus), sunflower oil (Helianthus annuus), soybean oil (Glycine max), peanut oil (Arachis hypogaea), sesame oil (Sesamum indicum), corn oil (Zea mays), coconut oil (Cocos nucifera), palm oil (Elaeis guineensis), palm kernel oil (Elaeis guineensis), avocado oil (Persea americana), grapeseed oil (Vitis vinifera), walnut oil (Juglans regia), hazelnut oil (Corylus avellana), almond oil (Prunus dulcis), macadamia nut oil (Macadamia integrifolia), pistachio oil (Pistacia vera), flaxseed oil (Linum usitatissimum), hemp seed oil (Cannabis sativa), pumpkin seed oil (Cucurbita pepo), rice bran oil (Oryza sativa), safflower oil (Carthamus tinctorius), mustard oil (Brassica juncea), camelina oil (Camelina sativa), cottonseed oil (Gossypium spp.), chia seed oil (Salvia hispanica), perilla oil (Perilla frutescens), moringa oil (Moringa oleifera), argan oil (Argania spinosa), pecan oil (Carya illinoinensis), medium chain triglycerides oil, vegetable oil, fish oil, butter, shortening, margarine, and lard.

A20. The composition of embodiment A16, wherein the at least a food-grade oil comprises a first extract of at least a psilocybin mushroom.

A21. The composition of embodiment A20, wherein the at least a psilocybin mushroom belongs to one or more genera selected from a group consisting of Psilocybe, Gymnopilus, Panacolus, Copelandia, Pluteus, Inocybe, Pholiotina, and Galerina.

A22. The composition of embodiment A16, wherein the at least a food-grade oil comprises a second extract of a mushroom of Hericium genus.

A23. The composition of embodiment A22, wherein the Hericium genus includes one or more species selected from a group consisting of Hericium erinaceus, Hericium americanum, Hericium coralloides, Hericium abietis, Hericium alpestre, Hericium cirrhatum, Hericium rajendrae, and Hericium flagellum.

A24. The composition of embodiment A1, wherein the composition further comprises an antioxidant, vitamin A, or zinc.

A25. The composition of embodiment A1, wherein the composition further comprises at least an emulsifier.

A26. A method of producing a composition of matter for treating at least a medical condition pertaining to a subject; the method comprising:
providing a plurality of active ingredients, comprising:
   providing a first extract comprising a first dosage of at least a psilocybin-based compound;
   providing a second extract comprising a second dosage of lion's mane; and
   providing a third dosage of cilostazol;
combining the first extract and the second extract to form a first mixture;
homogenizing the first mixture; and
adding the third dosage of cilostazol to the first mixture to form a second mixture, thereby producing the composition of matter.

A27. The method of embodiment A26, wherein providing the first extract comprises:
A) providing a first raw material comprising the at least a psilocybin-based compound;
B) providing a food-grade oil at a first temperature;
C) adding at least a portion of the first raw material to the food-grade oil to form a first raw material-food-grade oil mixture;
D) heating the first raw material-food-grade oil mixture to a second temperature for a first time period;
E) freezing the first raw material-food-grade oil mixture into a solid, frozen biomass;
F) heating the solid, frozen biomass at a third temperature for a second time period, thereby releasing the at least a psilocybin-based compound from the first raw material into the food-grade oil; and
G) straining the first raw material-food-grade oil mixture to separate the first raw material from the food-grade oil and yield the first extract.

A28. The method of embodiment A27, wherein providing the first extract further comprises:
H) separating an aqueous phase from an oil phase of the first extract.

A29. The method of embodiment A28, wherein separating the aqueous phase comprises:
resting the first extract for one to three hours until the first extract separates into an aqueous layer and an oil layer; and
separating the aqueous layer from the oil layer.

A30. The method of embodiment A28, wherein separating the aqueous phase comprises:
cooling the first extract to below 32° F., thereby freezing the aqueous phase into an ice layer; and
separating the ice layer from a rest of the first extract.

A31. The method of embodiment A27, the method further comprising:
mixing at least a portion of the separated first raw material from step G) with additional food-grade oil to form a recycled first raw material-food-grade oil mixture; and combining the recycled first raw material-food-grade oil mixture with the first raw material-food-grade oil mixture in step C).

A32. The method of embodiment A27, wherein the food-grade oil comprises one or more members selected from a group consisting of olive oil (Olea europaea), canola oil (Brassica napus), sunflower oil (Helianthus annuus), soybean oil (Glycine max), peanut oil (Arachis hypogaea), sesame oil (Sesamum indicum), corn oil (Zea mays), coconut oil (Cocos nucifera), palm oil (Elaeis guineensis), palm kernel oil (Elaeis guineensis), avocado oil (Persea americana), grapeseed oil (Vitis vinifera), walnut oil (Juglans regia), hazelnut oil (Corylus avellana), almond oil (Prunus dulcis), macadamia nut oil (Macadamia integrifolia), pistachio oil (Pistacia vera), flaxseed oil (Linum usitatissimum), hemp seed oil (Cannabis sativa), pumpkin seed oil (Cucurbita pepo), rice bran oil (Oryza sativa), safflower oil (Carthamus tinctorius), mustard oil (Brassica juncea), camelina oil (Camelina sativa), cottonseed oil (Gossypium spp.), chia seed oil (Salvia hispanica), perilla oil (Perilla frutescens), moringa oil (Moringa oleifera), argan oil (Argania spinosa), pecan oil (Carya illinoinensis), medium chain triglycerides oil, vegetable oil, fish oil, butter, shortening, margarine, and lard.

A33. The method of embodiment A27, wherein the first raw material comprises at least a psilocybin mushroom.

A34. The method of embodiment A33, wherein the at least a psilocybin mushroom belongs to one or more genera selected from a group consisting of Psilocybe, Gymnopilus, Panaeolus, Copelandia, Pluteus, Inocybe, Pholiotina, and Galerina.

A35. The method of embodiment A27, wherein the first temperature is between 200° F.-240° F.

A36. The method of embodiment A35, wherein the first temperature is approximately 225° F.

A37. The method of embodiment A27, wherein the second temperature is between 200° F.-240° F.

A38. The method of embodiment A37, wherein the second temperature is approximately 225° F.

A39. The method of embodiment A27, wherein the third temperature is between 175° F.-200° F.

A40. The method of embodiment A39, wherein the third temperature is approximately 185° F.

A41. The method of embodiment A27, wherein the first time period is between 4 hours and 9 hours.

A42. The method of embodiment A41, wherein the first time period is approximately 6 hours.

A43. The method of embodiment A27, wherein the second time period is between 2 hours and 4 hours.

A44. The method of embodiment A43, wherein the second time period is approximately 3 hours.

A45. The method of embodiment A26, wherein providing the second extract comprises:
A) providing a second raw material comprising lion's mane;
B) providing a food-grade oil at a fourth temperature;
C) adding at least a portion of the second raw material to the food-grade oil to form a second raw material-food-grade oil mixture;
D) heating the second raw material-food-grade oil mixture to a fifth temperature for a third time period;
E) freezing the second raw material-food-grade oil mixture into a solid, frozen biomass;
F) heating the solid, frozen biomass at a sixth temperature for a fourth time period, thereby releasing lion's mane from the second raw material into the food-grade oil; and
G) straining the second raw material-food-grade oil mixture to separate the second raw material from the food-grade oil and yield the second extract.

A46. The method of embodiment A45, wherein providing the second extract further comprises:
H) separating an aqueous phase from an oil phase of the second extract.

A47. The method of embodiment A46, wherein separating the aqueous phase comprises:
resting the second extract for one to three hours until the second extract separates into an aqueous layer and an oil layer; and
separating the aqueous layer from the oil layer.

A48. The method of embodiment A47, wherein separating the aqueous phase comprises:
cooling the second extract to below 32° F., thereby freezing the aqueous phase into an ice layer; and
separating the ice layer from a rest of the second extract.

A49. The method of embodiment A45, the method further comprising:
mixing at least a portion of the separated second raw material from step G) with additional food-grade oil to form a recycled second raw material-food-grade oil mixture; and
combining the recycled second raw material-food-grade oil mixture with the second raw material-food-grade oil mixture in step C).

A50. The method of embodiment A45, wherein the food-grade oil comprises one or more members selected from a group consisting of olive oil (Olea europaea), canola oil (Brassica napus), sunflower oil (Helianthus annuus), soybean oil (Glycine max), peanut oil (Arachis hypogaea), sesame oil (Sesamum indicum), corn oil (Zea mays), coconut oil (Cocos nucifera), palm oil (Elaeis guineensis), palm kernel oil (Elaeis guineensis), avocado oil (Persea americana), grapeseed oil (Vitis vinifera), walnut oil (Juglans regia), hazelnut oil (Corylus avellana), almond oil (Prunus dulcis), macadamia nut oil (Macadamia integrifolia), pistachio oil (Pistacia vera), flaxseed oil (Linum usitatissimum), hemp seed oil (Cannabis sativa), pumpkin seed oil (Cucurbita pepo), rice bran oil (Oryza sativa), safflower oil (Carthamus tinctorius), mustard oil (Brassica juncea), camelina oil (Camelina sativa), cottonseed oil (Gossypium spp.), chia seed oil (Salvia hispanica), perilla oil (Perilla frutescens), moringa oil (Moringa oleifera), argan oil (Argania spinosa), pecan oil (Carya illinoinensis), medium chain triglycerides oil, vegetable oil, fish oil, butter, shortening, margarine, and lard.

A51. The method of embodiment A45, wherein the second raw material comprises a mushroom of Hericium genus.

A52. The method of embodiment A51, wherein the Hericium genus includes one or more species selected from a group consisting of Hericium erinaceus, Hericium americanum, Hericium coralloides, Hericium abietis, Hericium alpestre, Hericium cirrhatum, Hericium rajendrae, and Hericium flagellum.

A53. The method of embodiment A45, wherein the fourth temperature is between 200° F.-240° F.

A54. The method of embodiment A53, wherein the fourth temperature is approximately 225° F.

A55. The method of embodiment A45, wherein the fifth temperature is between 200° F.-240° F.

A56. The method of embodiment A55, wherein the fifth temperature is approximately 225° F.

A57. The method of embodiment A45, wherein the sixth temperature is between 175° F.-200° F.

A58. The method of embodiment A57, wherein the sixth temperature is approximately 185° F.

A59. The method of embodiment A45, wherein the third time period is between 4 hours and 9 hours.

A60. The method of embodiment A59, wherein the third time period is approximately 6 hours.

A61. The method of embodiment A45, wherein the fourth time period is between 2 hours and 4 hours.

A62. The method of embodiment A61, wherein the fourth time period is approximately 3 hours.

A63. The method of embodiment A26, wherein the at least a psilocybin-based compound comprises psilocybin or psilocin.

A64. The method of embodiment A26, wherein the first dosage of at least a psilocybin-based compound is between 0.05 mg and 10 mg.

A65. The method of embodiment A64, wherein the first dosage of at least a psilocybin-based compound is approximately 0.5 mg.

A66. The method of embodiment A26, wherein the composition comprises between 0.1% and 99.7% by weight of the first dosage of at least a psilocybin-based compound.

A67. The method of embodiment A26, wherein the second dosage of lion's mane is between 5 mg and 1500 mg.

A68. The method of embodiment A67, wherein the second dosage of lion's mane is approximately 250 mg.

A69. The method of embodiment A26, wherein the composition comprises between 0.1% and 99.7% by weight of the second dosage of lion's mane.

A70. The method of embodiment A26, wherein the third dosage of cilostazol is between 25 mg and 100 mg.

A71. The method of embodiment A70, wherein the third dosage of cilostazol is approximately 50 mg.

A72. The method of embodiment A26, wherein the composition comprises between 0.1% and 99.7% by weight of the third dosage of cilostazol.

A73. The method of embodiment A26, further comprising combining the plurality of active ingredients with a matrix, wherein the composition comprises between 0.1% and 99.7% by weight of the matrix.

A74. The method of embodiment A73, wherein the matrix includes one or more members selected from a group consisting of food-grade oil, lactose, microcrystalline cellulose, starch, calcium phosphate, calcium carbonate, sucrose, maltodextrin, mannitol, sorbitol, and sodium chloride.

A75. The method of embodiment A73, wherein the method further comprises forming the composition into one or more of a tablet or a capsule form.

A76. The method of embodiment A26, wherein the method further comprises infusing an edible using the second mixture.

A77. The method of embodiment A26, wherein the third dosage of cilostazol is added in a powdered form.

A78. The method of embodiment A26, wherein the method further comprises combining the plurality of active ingredients with an antioxidant, vitamin A, or zinc.

A79. The method of embodiment A26, wherein the method further comprises combining the plurality of active ingredients with at least an emulsifier.

A80. A method for treating at least a medical condition pertaining to a subject, the method comprises administering to the subject a composition of matter, the composition comprising:
a plurality of active ingredients, wherein the plurality of active ingredients comprises:
   a first dosage of at least a psilocybin-based compound;
   a second dosage of lion's mane; and
   a third dosage of cilostazol.

A81. The method of embodiment A80, wherein the at least a psilocybin-based compound comprises psilocybin or psilocin.

A82. The method of embodiment A80, wherein the first dosage of at least a psilocybin-based compound is between 0.05 mg and 10 mg.

A83. The method of embodiment A82, wherein the first dosage of at least a psilocybin-based compound is approximately 0.5 mg.

A84. The method of embodiment A80, wherein the composition comprises between 0.1% and 99.7% by weight of the first dosage of at least a psilocybin-based compound.

A85. The method of embodiment A80, wherein the second dosage of lion's mane is between 5 mg and 1500 mg.

A86. The method of embodiment A85, wherein the second dosage of lion's mane is approximately 250 mg.

A87. The method of embodiment A80, wherein the composition comprises between 0.1% and 99.7% by weight of the second dosage of lion's mane.

A88. The method of embodiment A80, wherein the third dosage of cilostazol is between 25 mg and 100 mg.

A89. The method of embodiment A88, wherein the third dosage of cilostazol is approximately 50 mg.

A90. The method of embodiment A80, wherein the composition comprises between 0.1% and 99.7% by weight of the third dosage of cilostazol.

A91. The method of embodiment A80, wherein administering the composition of matter comprises administering the composition of matter in one or more of a tablet form or a capsule form.

A92. The method of embodiment A80, wherein administering the composition of matter comprises providing to the subject an edible comprising the composition of matter.

A93. The method of embodiment A80, wherein administering the composition of matter comprises administering the composition of matter three times a week.

A94. The method of embodiment A80, wherein administering the composition of matter comprises administering the composition of matter once a week.

A95. The method of embodiment A80, wherein administering the composition of matter comprises administering the composition of matter on a daily basis.

A96. The method of embodiment A80, wherein composition of matter is administered at a frequency between three times a day and once every two weeks.

A97. The method of embodiment A80, wherein the at least a medical condition comprises one or more members selected from a group consisting of neurological condition, macular degeneration, retinal conditions, depression, concussion, pain including pain from neuroischemic disorder, anxiety, post-traumatic stress disorder (PTSD), cluster headaches, addiction, smoking cessation, obsessive compulsive disorder (OCD), attention deficit disorder (ADD), small blood vessel disease, Alzheimer's disease, dementia, Parkinson's disease, Huntington's disease, motor neuron disease, spinal muscular atrophy, stroke, spine injury, brain injury, peripheral neuropathy, fibromyalgia, migraine, menopause, weight loss, cancer, multiple sclerosis, hypoxia-ischemia injury, psychosis, nausea, inflammatory disease, rheumatoid arthritis, infection, inflammatory bowel disease, cardiovascular condition, and diabetic complication including diabetic polyneuropathy.

A98. The method of embodiment A80, the method further comprising achieving within the subject one or more benefits selected from a group consisting of increasing neuroplasticity, accelerating growth of brain cells, and enhancing mental function.

A99. A composition of matter for treating at least a medical condition pertaining to a subject, wherein the composition is produced by:
providing a plurality of active ingredients, comprising:
   providing a first extract comprising a first dosage of at least a psilocybin-based compound;
   providing a second extract comprising a second dosage of lion's mane; and
   providing a third dosage of cilostazol;
combining the first extract and the second extract to form a first mixture;
homogenizing the first mixture; and
adding the third dosage of cilostazol to the first mixture to form a second mixture, thereby producing the composition of matter.

A100. The composition of embodiment A99, wherein providing the first extract comprises:
A) providing a first raw material comprising the at least a psilocybin-based compound;
B) providing a food-grade oil at a first temperature;
C) adding at least a portion of the first raw material to the food-grade oil to form a first raw material-food-grade oil mixture;
D) heating the first raw material-food-grade oil mixture to a second temperature for a first time period;
E) freezing the first raw material-food-grade oil mixture into a solid, frozen biomass;
F) heating the solid, frozen biomass at a third temperature for a second time period, thereby releasing the at least a psilocybin-based compound from the first raw material into the food-grade oil; and
G) straining the first raw material-food-grade oil mixture to separate the first raw material from the food-grade oil and yield the first extract.

A101. The composition of embodiment A100, wherein providing the first extract further comprises:
H) separating an aqueous phase from an oil phase of the first extract.

A102. The composition of embodiment A101, wherein separating the aqueous phase comprises:
resting the first extract for one to three hours until the first extract separates into an aqueous layer and an oil layer; and
separating the aqueous layer from the oil layer.

A103. The composition of embodiment A101, wherein separating the aqueous phase comprises:
cooling the first extract to below 32° F., thereby freezing the aqueous phase into an ice layer; and
separating the ice layer from a rest of the first extract.

A104. The composition of embodiment A100, a production of which further comprising:
mixing at least a portion of the separated first raw material from step G) with additional food-grade oil to form a recycled first raw material-food-grade oil mixture; and
combining the recycled first raw material-food-grade oil mixture with the first raw material-food-grade oil mixture in step C).

A105. The composition of embodiment A100, wherein the food-grade oil comprises one or more members selected from a group consisting of olive oil (Olea europaea), canola oil (Brassica napus), sunflower oil (Helianthus annuus), soybean oil (Glycine max), peanut oil (Arachis hypogaea), sesame oil (Sesamum indicum), corn oil (Zea mays), coconut oil (Cocos nucifera), palm oil (Elaeis guineensis), palm kernel oil (Elaeis guineensis), avocado oil (Persea americana), grapeseed oil (Vitis vinifera), walnut oil (Juglans regia), hazelnut oil (Corylus avellana), almond oil (Prunus dulcis), macadamia nut oil (Macadamia integrifolia), pistachio oil (Pistacia vera), flaxseed oil (Linum usitatissimum), hemp seed oil (Cannabis sativa), pumpkin seed oil (Cucurbita pepo), rice bran oil (Oryza sativa), safflower oil (Carthamus tinctorius), mustard oil (Brassica juncea), camelina oil (Camelina sativa), cottonseed oil (Gossypium spp.), chia seed oil (Salvia hispanica), perilla oil (Perilla frutescens), moringa oil (Moringa oleifera), argan oil (Argania spinosa), pecan oil (Carya illinoinensis), medium chain triglycerides oil, vegetable oil, fish oil, butter, shortening, margarine, and lard.

A106. The composition of embodiment A100, wherein the first raw material comprises at least a psilocybin mushroom.

A107. The composition of embodiment A106, wherein the at least a psilocybin mushroom belongs to one or more genera selected from a group consisting of Psilocybe, Gymnopilus, Panaeolus, Copelandia, Pluteus, Inocybe, Pholiotina, and Galerina.

A108. The composition of embodiment A100, wherein the first temperature is between 200° F.-240° F.

A109. The composition of embodiment A108, wherein the first temperature is approximately 225° F.

A110. The composition of embodiment A100, wherein the second temperature is between 200° F.-240° F.

A111. The composition of embodiment A110, wherein the second temperature is approximately 225° F.

A112. The composition of embodiment A100, wherein the third temperature is between 175° F.-200° F.

A113. The composition of embodiment A112, wherein the third temperature is approximately 185° F.

A114. The composition of embodiment A100, wherein the first time period is between 4 hours and 9 hours.

A115. The composition of embodiment A114, wherein the first time period is approximately 6 hours.

A116. The composition of embodiment A100, wherein the second time period is between 2 hours and 4 hours.

A117. The composition of embodiment A116, wherein the second time period is approximately 3 hours.

A118. The composition of embodiment A99, wherein providing the second extract comprises:
A) providing a second raw material comprising lion's mane;
B) providing a food-grade oil at a fourth temperature;
C) adding at least a portion of the second raw material to the food-grade oil to form a second raw material-food-grade oil mixture;
D) heating the second raw material-food-grade oil mixture to a fifth temperature for a third time period;
E) freezing the second raw material-food-grade oil mixture into a solid, frozen biomass;
F) heating the solid, frozen biomass at a sixth temperature for a fourth time period, thereby releasing lion's mane from the second raw material into the food-grade oil; and
G) straining the second raw material-food-grade oil mixture to separate the second raw material from the food-grade oil and yield the second extract.

A119. The composition of embodiment A118, wherein providing the second extract further comprises:
H) separating an aqueous phase from an oil phase of the second extract.

A120. The composition of embodiment A119, wherein separating the aqueous phase comprises:
resting the second extract for one to three hours until the second extract separates into an aqueous layer and an oil layer; and
separating the aqueous layer from the oil layer.

A121. The composition of embodiment A119, wherein separating the aqueous phase comprises:
cooling the second extract to below 32° F., thereby freezing the aqueous phase into an ice layer; and
separating the ice layer from a rest of the second extract.

A122. The composition of embodiment A118, a production of which further comprises:
mixing at least a portion of the separated second raw material from step G) with additional food-grade oil to form a recycled second raw material-food-grade oil mixture; and
combining the recycled second raw material-food-grade oil mixture with the second raw material-food-grade oil mixture in step C).

A123. The composition of embodiment A118, wherein the food-grade oil comprises one or more members selected from a group consisting of olive oil (Olea europaea), canola oil (Brassica napus), sunflower oil (Helianthus annuus), soybean oil (Glycine max), peanut oil (Arachis hypogaea), sesame oil (Sesamum indicum), corn oil (Zea mays), coconut oil (Cocos nucifera), palm oil (Elaeis guineensis), palm kernel oil (Elaeis guineensis), avocado oil (Persea americana), grapeseed oil (Vitis vinifera), walnut oil (Juglans regia), hazelnut oil (Corylus avellana), almond oil (Prunus dulcis), macadamia nut oil (Macadamia integrifolia), pistachio oil (Pistacia vera), flaxseed oil (Linum usitatissimum), hemp seed oil (Cannabis sativa), pumpkin seed oil (Cucurbita pepo), rice bran oil (Oryza sativa), safflower oil (Carthamus tinctorius), mustard oil (Brassica juncea), camelina oil (Camelina sativa), cottonseed oil (Gossypium spp.), chia seed oil (Salvia hispanica), perilla oil (Perilla frutescens), moringa oil (Moringa oleifera), argan oil (Argania spinosa), pecan oil (Carya illinoinensis), medium chain triglycerides oil, vegetable oil, fish oil, butter, shortening, margarine, and lard.

A124. The composition of embodiment A118, wherein the second raw material comprises a mushroom of Hericium genus.

A125. The composition of embodiment A124, wherein the Hericium genus includes one or more species selected from a group consisting of Hericium erinaceus, Hericium americanum, Hericium coralloides, Hericium abietis, Hericium alpestre, Hericium cirrhatum, Hericium rajendrae, and Hericium flagellum.

A126. The composition of embodiment A118, wherein the fourth temperature is between 200° F.-240° F.

A127. The composition of embodiment A126, wherein the fourth temperature is approximately 225° F.

A128. The composition of embodiment A118, wherein the fifth temperature is between 200° F.-240° F.

A129. The composition of embodiment A128, wherein the fifth temperature is approximately 225° F.

A130. The composition of embodiment A118, wherein the sixth temperature is between 175° F.-200° F.

A131. The composition of embodiment A130, wherein the sixth temperature is approximately 185° F.

A132. The composition of embodiment A118, wherein the third time period is between 4 hours and 9 hours.

A133. The composition of embodiment A132, wherein the third time period is approximately 6 hours.

A134. The composition of embodiment A118, wherein the fourth time period is between 2 hours and 4 hours.

A135. The composition of embodiment A134, wherein the fourth time period is approximately 3 hours.

A136. The composition of embodiment A99, wherein the at least a psilocybin-based compound comprises psilocybin or psilocin.

A137. The composition of embodiment A99, wherein the first dosage of at least a psilocybin-based compound is between 0.05 mg and 10 mg.

A138. The composition of embodiment A137, wherein the first dosage of at least a psilocybin-based compound is approximately 0.5 mg.

A139. The composition of embodiment A99, wherein the composition comprises between 0.1% and 99.7% by weight of the first dosage of at least a psilocybin-based compound.

A140. The composition of embodiment A99, wherein the second dosage of lion's mane is between 5 mg and 1500 mg.

A141. The composition of embodiment A140, wherein the second dosage lion's mane is approximately 250 mg.

A142. The composition of embodiment A99, wherein the composition comprises between 0.1% and 99.7% by weight of the second dosage of lion's mane.

A143. The composition of embodiment A99, wherein the third dosage of cilostazol is between 25 mg and 100 mg.

A144. The composition of embodiment A143, wherein the third dosage of cilostazol is approximately 50 mg.

A145. The composition of embodiment A99, wherein the composition comprises between 0.1% and 99.7% by weight of the third dosage of cilostazol.

A146. The composition of embodiment A99, a production of which further comprises incorporating the plurality of active ingredients in a matrix, wherein the composition comprises between 0.1% and 99.7% by weight of the matrix.

A147. The composition of embodiment A146, wherein the matrix includes one or more members selected from a group consisting of food-grade oil, lactose, microcrystalline cellulose, starch, calcium phosphate, calcium carbonate, sucrose, maltodextrin, mannitol, sorbitol, and sodium chloride.

A148. The composition of embodiment A146, the production of which further comprises forming the composition into one or more of a tablet or a capsule form.

A149. The composition of embodiment A99, a production of which further comprises infusing an edible using the second mixture.

A150. The composition of embodiment A99, wherein the third dosage of cilostazol is added in a powdered form.

A151. The composition of embodiment A99, a production of which further comprises combining the plurality of active ingredients with an antioxidant, vitamin A, or zinc.

A152. The composition of embodiment A99, a production of which further comprises combining the plurality of active ingredients with at least an emulsifier.

Exemplary embodiments have been disclosed above and illustrated in the accompanying drawings. It will be understood by those skilled in the art that various changes, omissions and additions may be made to that which is specifically disclosed herein without departing from the spirit and scope of the present invention.

What is claimed is:

1. A method of producing a composition of matter for treating at least a medical condition pertaining to a subject; the method comprising:
providing a plurality of active ingredients, comprising:
providing a first extract comprising a first dosage of at least a psilocybin-based compound;
providing a second extract comprising a second dosage of lion's mane; and
providing a third dosage of cilostazol;
combining the first extract and the second extract to form a first mixture;
homogenizing the first mixture; and
adding the third dosage of cilostazol to the first mixture to form a second mixture, thereby producing the composition of matter.

2. The method of claim 1, wherein providing the first extract comprises:
A) providing at least a psilocybin mushroom comprising the at least a psilocybin-based compound;
B) providing a food-grade oil at a temperature between 200° F.-240° F.;
C) adding at least a portion of the at least a psilocybin mushroom to the food-grade oil to form a psilocybin mushroom-food-grade oil mixture;
D) heating the psilocybin mushroom-food-grade oil mixture to a temperature between 200° F.-240° F. for between 4 hours and 9 hours;
E) freezing the psilocybin mushroom-food-grade oil mixture into a solid, frozen biomass;
F) heating the solid, frozen biomass at a temperature between 175° F.-200° F. for between 2 hours and 4 hours, thereby releasing the at least a psilocybin-based compound from the psilocybin mushroom into the food-grade oil; and
G) straining the first raw material-food-grade oil mixture to separate the psilocybin mushroom from the food-grade oil and yield the first extract.

3. The method of claim 2, wherein providing the first extract further comprises:
H) separating an aqueous phase from an oil phase of the first extract.

4. The method of claim 3, wherein separating the aqueous phase comprises:
resting the first extract for one to three hours until the first extract separates into an aqueous layer and an oil layer; and
separating the aqueous layer from the oil layer.

5. The method of claim 3, wherein separating the aqueous phase comprises:
cooling the first extract to below 32° F., thereby freezing the aqueous phase into an ice layer; and
separating the ice layer from a rest of the first extract.

6. The method of claim 3, the method further comprising:
I) mixing at least a portion of the separated psilocybin mushroom from step G) with additional food-grade oil to form a recycled psilocybin mushroom-food-grade oil mixture; and
J) combining the recycled psilocybin mushroom-food-grade oil mixture with the psilocybin mushroom-food-grade oil mixture in step C).

7. The method of claim 2, wherein the food-grade oil comprises one or more members selected from a group consisting of olive oil (Olea europaea), canola oil (Brassica napus), sunflower oil (Helianthus anmuus), soybean oil (Glycine max), peanut oil (Arachis hypogaea), sesame oil (Sesamum indicum), corn oil (Zea mays), coconut oil (Cocos nucifera), palm oil (Elaeis guineensis), palm kernel oil (Elaeis guineensis), avocado oil (Persea americana), grapeseed oil (Vitis vinifera), walnut oil (Juglans regia), hazelnut oil (Corylus avellana), almond oil (Prunus dulcis), macadamia nut oil (Macadamia integrifolia), pistachio oil (Pistacia vera), flaxseed oil (Linum usitatissimum), hemp seed oil (Cannabis sativa), pumpkin seed oil (Cucurbita pepo), rice bran oil (Oryza sativa), safflower oil (Carthamus tinctorius), mustard oil (Brassica juncea), camelina oil (Camelina sativa), cottonseed oil (Gossypium spp.), chia seed oil (Salvia hispanica), perilla oil (Perilla frutescens), moringa oil (Moringa oleifera), argan oil (Argania spinosa), pecan oil (Carya illinoinensis), medium chain triglycerides oil, vegetable oil, fish oil, butter, shortening, margarine, and lard.

8. The method of claim 2, wherein the at least a psilocybin mushroom belongs to a genus selected from the group consisting of Psilocybe, Gymnopilus, Panaeolus, Copelandia, Pluteus, Inocybe, Pholiotina, and Galerina.

9. The method of claim 1, wherein the first dosage of at least a psilocybin-based compound is between 0.1 mg and 10 mg.

10. The method of claim 1, wherein the second dosage of lion's mane is between 5 mg and 1500 mg.

11. The method of claim 1, wherein the third dosage of cilostazol is between 25 mg and 200 mg.

12. The method of claim 1, wherein providing the second extract comprises:
A) providing a mushroom of Hericium genus comprising lion's mane;
B) providing a food-grade oil at a temperature between 200° F.-240° F.;
C) adding at least a portion of the mushroom of Hericium genus to the food-grade oil to form a mushroom of Hericium genus-food-grade oil mixture;
D) heating the mushroom of Hericium genus-food-grade oil mixture to a temperature between 200° F.-240° F. for between 4 hours and 9 hours;
E) freezing the mushroom of Hericium genus-food-grade oil mixture into a solid, frozen biomass;
F) heating the solid, frozen biomass at a temperature between 175° F.-200° F. for between 2 hours and 4 hours, thereby releasing lion's mane from the mushroom of Hericium genus into the food-grade oil; and
G) straining the mushroom of Hericium genus-food-grade oil mixture to separate the mushroom of Hericium genus from the food-grade oil and yield the second extract.

13. The method of claim 12, wherein providing the second extract further comprises:
H) separating an aqueous phase from an oil phase of the second extract.

14. The method of claim 13, wherein separating the aqueous phase comprises:
resting the second extract for one to three hours until the second extract separates into an aqueous layer and an oil layer; and
separating the aqueous layer from the oil layer.

15. The method of claim 13, wherein separating the aqueous phase comprises:
cooling the second extract to below 32° F., thereby freezing the aqueous phase into an ice layer; and
separating the ice layer from a rest of the second extract.

16. The method of claim 12, the method further comprising:
I) mixing at least a portion of the separated mushroom of Hericium genus from step G) with additional food-grade oil to form a recycled mushroom of Hericium genus-food-grade oil mixture; and
J) combining the recycled mushroom of Hericium genus-food-grade oil mixture with the mushroom of Hericium genus-food-grade oil mixture in step C).

17. The method of claim 12, wherein the mushroom of Hericium genus comprises a mushroom of a species selected from the group consisting of Hericium erinaceus, Hericium americanum, Hericium coralloides, Hericium abietis, Hericium alpestre, Hericium cirrhatum, Hericium rajendrae, and Hericium flagellum.

18. The method of claim 1, wherein the third dosage of cilostazol is added in a powdered form.

19. The method of claim 1, wherein the method further comprises forming the composition of matter into one or more of a tablet or a capsule form.

20. The method of claim 1, wherein the method further comprises infusing an edible using the second mixture.
